SEQUENCE LISTING (16 sequences)

SEQ ID NO: 1 - DNA, 20 bp, Artificial Sequence
  Synthetic oligonucleotide
agagtttgatcctggctcag

SEQ ID NO: 2 - DNA, 19 bp, Artificial Sequence
  Synthetic oligonucleotide
ggttaccttgttacgactt

SEQ ID NO: 3 - DNA, 21 bp, Artificial Sequence
  Synthetic oligonucleotide
catttgttcacgaggctttcc

SEQ ID NO: 4 - DNA, 26 bp, Artificial Sequence
  Synthetic oligonucleotide
gtttttccagttagcttccttcatgt

SEQ ID NO: 5 - DNA, 23 bp, Artificial Sequence
  Synthetic oligonucleotide
tgtgtatcccacaaggtttcaga

SEQ ID NO: 6 - DNA, 24 bp, Artificial Sequence
  Synthetic oligonucleotide
ttattaccctctcctcctcaagca

SEQ ID NO: 7 - DNA, 17 bp, Artificial Sequence
  Synthetic oligonucleotide
cgcagcacgagcaggat

SEQ ID NO: 8 - DNA, 23 bp, Artificial Sequence
  Synthetic oligonucleotide
ccaggatcaagatgcaaagaatg

SEQ ID NO: 9 - DNA, 19 bp, Artificial Sequence
  Synthetic oligonucleotide
tgccccttcaatggttggt

SEQ ID NO: 10 - DNA, 18 bp, Artificial Sequence
  Synthetic oligonucleotide
tccttcggcccacttcct

SEQ ID NO: 11 - DNA, 21 bp, Artificial Sequence
  Synthetic oligonucleotide
ccttcctcttcctcaggcaat

SEQ ID NO: 12 - DNA, 27 bp, Artificial Sequence
  Synthetic oligonucleotide
taattctctctccacttcagaaatcct

SEQ ID NO: 13 - DNA, 21 bp, Artificial Sequence
  Synthetic oligonucleotide
atcagtgggaacaggctcaga

SEQ ID NO: 14 - DNA, 28 bp, Artificial Sequence
  Synthetic oligonucleotide
ttgtccactagactattgaccttctttg

SEQ ID NO: 15 - DNA, 24 bp, Artificial Sequence
  Synthetic oligonucleotide
ttggttcgccactctaatcagtag

SEQ ID NO: 16 - DNA, 22 bp, Artificial Sequence
  Synthetic oligonucleotide
gcagttcagccacactctcact

(19) United States
(12) Patent Application Publication
(10) Pub. No.: US 2015/0224152 A1
(43) Pub. Date: Aug. 13, 2015

(54) METHODS FOR MODULATING BACTERIAL INFECTION

(71) Applicants: Dan Littman, New York, NY (US); Ivaylo Ivanov, New York, NY (US); Eoin Brodie, Piedmont, CA (US); Yoshinori Umesaki, Tokyo (JP); Kenya Honda, Toyko (JP)

(72) Inventors: Dan Littman, New York, NY (US); Ivaylo Ivanov, New York, NY (US); Eoin Brodie, Piedmont, CA (US); Yoshinori Umesaki, Tokyo (JP); Kenya Honda, Toyko (JP)

(21) Appl. No.: 14/526,911

(22) Filed: Oct. 29, 2014

Related U.S. Application Data
(62) Division of application No. 13/501,615, filed on Jul. 19, 2012, now abandoned.
(60) Provisional application No. 61/279,152, filed on Oct. 15, 2009.

(51) Int. Cl. A61K 35/74; C12Q 1/68

(52) U.S. Cl. CPC A61K 35/74; C12Q 1/6881; C12Q 2600/16; C12Q 2600/158

(57) ABSTRACT

The present invention relates to methods for treating or reducing the risk of or preventing diseases caused by or associated with pathogenic bacteria. More particularly, the present invention relates to methods for treating or reducing the risk of or preventing diseases caused by or associated with pathogenic bacteria of the gastrointestinal (GI) tract. The present invention further relates to methods for promoting pathways induced by commensal bacteria of the GI tract that lead to Th17 differentiation, which in turn leads to localized and systemic accumulation of Th17 cells. Compositions and medicaments are also described herein that are used alleviate and/or prevent symptoms associated with diseases caused by or associated with pathogenic bacteria. Accordingly, the compositions, medicaments and methods described herein may be used to address the needs of patients or subjects that would benefit from increased Th17 cell differentiation.

### CROSS REFERENCE TO RELATED APPLICATIONS

The present application is a Divisional Application claiming the priority of co-pending U.S. application Ser. No. 13/501,615 filed Jul. 19, 2012, which in turn claims priority from PCT Application No. PCT/US2010/002757 filed Oct. 15, 2010, which in turn claims priority from U.S. Provisional Application No. 61/279,152, filed Oct. 15, 2009. Applicants claim the benefits of 35 U.S.C. Section 120 as to the said U.S. and PCT applications and priority under 35 U.S.C. Section 119 as to the said U.S. Provisional application, and the entire disclosure of each of these applications is incorporated herein by reference in its entirety.

### GOVERNMENTAL SUPPORT

The research leading to the present invention was funded in part by National Institutes of Health Grant Numbers R01AI080885 and AI33856 and under the auspices of the United States Department of Energy under contract DEAC02-05CH11231. Accordingly, the United States government has certain rights in the invention.

### FIELD OF THE INVENTION

The present invention relates to methods for treating or reducing the risk of or preventing diseases caused by or associated with pathogenic bacteria. More particularly, the present invention relates to methods for treating or reducing the risk of or preventing diseases caused by or associated with pathogenic bacteria of the gastrointestinal (GI) tract. The present invention further relates to methods for promoting pathways induced by commensal bacteria of the GI tract that lead to Th17 differentiation, which in turn leads to localized and systemic accumulation of Th17 cells.

### BACKGROUND OF THE INVENTION

Several publications and patent documents are referenced in this application in order to more fully describe the state of the art to which this invention pertains. The disclosure of each of these publications and documents is incorporated by reference herein.

The vertebrate intestine is typically colonized by hundreds of distinct species of microorganisms that have a mutually beneficial relationship with the host. Intestinal microbiota are known to influence the development and balance of the host immune system, and have been implicated in prevention of damage induced by opportunistic microbes, in repair of damage to the mucosal barrier, and in influencing systemic autoimmune diseases (Backhed et al., 2005; Macpherson and Harris, 2004; Rakoff-Nahoum and Medzhitov, 2006). CD4⁺ T cells acquire distinct functional properties in response to signals conveyed by commensal and pathogenic microbe-activated cells of the innate immune system (Seder and Paul, 1994). T-helper type 1 (Th1) and Th2 cells control intracellular microorganisms and helminths, respectively (Abbas et al., 1996; Glimcher and Murphy, 2000), whereas the induced regulatory T cells (iTreg) suppress excessive immune responses (Gavin and Rudensky, 2003). Th17 cells secrete IL-17, IL-17F, and IL-22, and have significant roles in protecting the host from bacterial and fungal infections, particularly at mucosal surfaces. Th17 cells also have potent inflammatory potential, and thus are key mediators of autoimmune disease (Aujla et al., 2007; Bettelli et al., 2007). Th17 and Treg cells are both dependent on TGF-β for their differentiation and are defined by the expression of the lineage-specific transcription factors RORγt and Foxp3, respectively (Fontenot et al., 2003; Hori et al., 2003; Ivanov et al., 2006; Khattri et al., 2003; Mangan et al., 2006) (Veldhoen et al., 2006). At appropriate concentrations of TGF-β and IL-6, antigen-activated CD4⁺ T cells up-regulate RORγt and express Th17 cell cytokines (Zhou et al., 2008).

Th17 cells are most abundant at steady state in gut-associated tissues, particularly the small intestinal lamina propria (SI LP) (Ivanov et al., 2008; Ivanov et al., 2006), where they accumulate only in the presence of luminal commensal microbiota (Atarashi et al., 2008; Hall et al., 2008; Ivanov et al., 2008). Germ-free (GF) mice, which lack Th17 cells in the SI LP (and also in the colon), acquire them following colonization with conventional microbiota. Treatment of newborn mice with antibiotics, particularly vancomycin, resulted in marked reduction in the number of Th17 cells in the SI LP. Most strikingly, C57BL/6 (B6) mice obtained from different commercial vendors displayed marked differences in the proportion of Th17 cells in the SI LP (Ivanov et al., 2008). Thus, mice from the Jackson Laboratory had very low numbers of SI LP Th17 cells compared to mice of the same strain obtained from Taconic Farms. Transfer into GF mice of intestinal contents of Taconic B6 mice, but not Jackson B6 mice, induced Th17 cell accumulation, and Jackson mice acquired Th17 cells within weeks of co-housing with mice from Taconic Farms. GF mice colonized only with a defined cocktail of bacteria (Altered Schaedler Flora, or ASF) lacked intestinal Th17 cells (Ivanov et al., 2008). These results demonstrated that the induction of Th17 cells in the SI LP is controlled not by the presence of bacteria per se, but by the composition of the intestinal microbiota and, presumably, the presence of specific bacterial taxa. Intriguingly, Treg cells, which, like Th17 cells, are abundant in the intestine, were increased in proportion in the SI LP in GF mice, and their numbers were inversely correlated to the proportion of Th17 cells. Signals derived from microbiota may thus influence the differentiation potential of multipotent CD4⁺ T cells in the lamina propria (Zhou et al., 2008).

### SUMMARY

The present disclosure is directed to the discovery that a population of a single species of bacteria or a component thereof can enhance mucosal immunity in a subject by increasing expression of at least one host molecule that is positively correlated with enhanced Th17 differentiation and enhanced mucosal immunity. Also encompassed are compositions, uses, and methods pertaining to a single species of bacteria or a component thereof and compositions, uses, and methods pertaining to at least one of the host molecules induced thereby.

Accordingly, in a first aspect, a method for enhancing mucosal immunity in a subject in need thereof is described, the method comprising administering a therapeutic amount of a single species of Th17 inducing bacteria or a component thereof to the subject. In an embodiment, the method further comprises measuring Th17 cell differentiation in the subject, wherein an increase in the Th17 cell differentiation in the subject after the administering relative to prior to the administering is a positive indicator of enhanced mucosal immunity.

In another aspect, a method for enhancing mucosal immunity in a subject in need thereof is described, the method comprising administering a therapeutic amount of a single species of bacteria or a component thereof to the subject, wherein an increase in at least one indicator of Th17 differentiation after the administering relative to prior to the administering is positively correlated with enhanced mucosal immunity.

In yet another aspect, a method for enhancing mucosal immunity in a subject in need thereof is described, the method comprising: a) administering a therapeutic amount of a single species of bacteria or a component thereof to the subject; and b) measuring Th17 cell differentiation in the subject, wherein an increase in the Th17 cell differentiation in the subject after the administering relative to prior to the administering is a positive indicator of enhanced mucosal immunity.

In an embodiment of the methods, the single species of Th17 inducing bacteria or single species of bacteria is segmented filamentous bacteria (SFB). In another embodiment of the methods, the bacteria is a homologue of SFB present in human microbiota. In yet another embodiment of the method, the bacteria is a human commensal species other than SFB. In a further embodiment, the bacteria is a spore formant such as a Clostridia spp. or a Bacillus spp.

In embodiments wherein the method further comprises measuring Th17 cell differentiation in the subject, an increase in Th17 cell differentiation or an increase in at least one indicator of Th17 differentiation in the subject after the administering relative to prior to the administering is a positive indicator of enhanced mucosal immunity in the subject. In an embodiment of the method, the increase in the Th17 cell differentiation or the increase in at least one indicator of Th17 differentiation is detected as an increase in Th17 cell number, Th17 activity, or expression of Th17 specific cytokines after the administering relative to the Th17 cell number, Th17 activity, or expression of Th17 specific cytokines determined prior to the administering. As described herein, the increase in Th17 activity or expression of Th17 specific cytokines is detected as an increase in expression of at least one of RORγt, IL-17A, IL-17F, IL-22, IL23, IL23R, CD161, and CCR6 after the administering relative to the expression of at least one of RORγt, IL-17A, IL-17F, IL-22, IL23, IL23R, CD161, and CCR6 determined prior to the administering. In an embodiment of the method, the Th17 cell number, Th17 activity, or expression of Th17 specific cytokines is measured in a blood sample or biopsy isolated from the subject after the administering and the increase is determined relative to the Th17 cell number, Th17 activity, or expression of Th17 specific cytokines determined in a blood sample or biopsy isolated from the subject prior to the administering.

In another embodiment, the single species of Th17 inducing bacteria or single species of bacteria promotes Th17 cell differentiation by proliferating, expressing a bacterial product, or attaching to intestinal epithelial cells in the subject. In a particular embodiment, the bacterial product may be a bacterial cell wall component.

In another embodiment, the subject in need of enhanced mucosal immunity is a patient infected with a pathogenic bacteria or at risk for infection with a pathogenic bacteria. In particular embodiment of the invention, the pathogenic bacteria is an antibiotic resistant pathogenic bacteria. Examples of antibiotic resistant pathogenic bacteria include: various strains of Staphylococcus aureus (e.g., methicillin-resistant Staphylococcus aureus; MRSA), Streptococcus pyogenes, Enterococcus faecium, Pseudomonas aeruginosa, Clostridium difficile, E. coli, Salmonella, and Acinetobacter baumannii.

In another aspect, a method for promoting Th17 differentiation in a subject in need thereof is described, the method comprising: a) administering a therapeutic amount of a population of segmented filamentous bacteria (SFB) or a component thereof, or at least one SFB induced host cell molecule to the subject; and optionally b) measuring Th17 cell activity in the subject, wherein an increase in the Th17 cell activity in the subject after the administering relative to prior to the administering is a positive indicator of enhanced Th17 differentiation.

In an embodiment wherein at least one SFB induced host cell molecule is administered to the subject, the at least one SFB induced host cell molecule is serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; or lymphocyte antigen 6 complex, locus D.

In a further embodiment, the at least one SFB induced host cell molecule is at least one of serum amyloid A 1, resistin like beta, or serum amyloid A 2. In a still further embodiment, the at least one SFB induced host cell molecule is a combination of two of serum amyloid A 1, resistin like beta, or serum amyloid A 2 or a combination of serum amyloid A 1, resistin like beta, and serum amyloid A 2. In yet another embodiment, the at least one SFB induced host cell molecule is at least one of serum amyloid A 1 or serum amyloid A 2. In a further embodiment, the at least one SFB induced host cell molecule is a combination of serum amyloid A 1 and serum amyloid A 2. In yet another further embodiment, the at least one SFB induced host cell molecule is resistin like beta, which may be used alone or in any and all combinations with other SFB induced host cell molecules as described herein.

In an embodiment of the method for promoting Th17 differentiation in a subject in need thereof, the increase in Th17 cell activity is detected as an increase in Th17 cell number, Th17 function, or expression of Th17 specific cytokines after the administering relative to the Th17 cell number, Th17 function, or expression of Th17 specific cytokines determined prior to the administering. As described herein, the increase in Th17 function or expression of Th17 specific cytokines is detected as an increase in expression of at least one of RORγt, IL-17A, IL-17F, IL-22, IL23, IL23R, CD161, and CCR6 after the administering relative to the expression of at least one of RORγt, IL-17A, IL-17F, IL-22, IL23, IL23R, CD161, and CCR6 determined prior to the administering. In an embodiment of the method, the Th17 cell activity is measured in a blood sample or biopsy isolated from the subject after the administering and the increase is determined relative to the Th17 cell activity determined in a blood sample or biopsy isolated from the subject prior to the administering.

In another embodiment, the population of segmented filamentous bacteria (SFB) or a component thereof, promotes Th17 cell differentiation by proliferating, expressing a bacterial product, or attaching to intestinal epithelial cells in the subject. In a particular embodiment, the bacterial product may be a bacterial cell wall component.

In another embodiment, the subject in need of enhanced Th17 differentiation is a subject or patient infected with a pathogenic bacteria or at risk for infection with a pathogenic bacteria. In particular embodiment of the invention, the pathogenic bacteria is an antibiotic resistant pathogenic bacteria. An exemplary type of pathogenic bacteria is an antibiotic resistant pathogenic bacteria. Other examples of antibiotic resistant pathogenic bacteria are known to skilled artisans and are described herein.

In another aspect, a method for identifying a compound that promotes Th17 differentiation is described, the method comprising: a) contacting a cell population comprising dendritic cells and naive CD4⁺ T cells with at least one of serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; or lymphocyte antigen 6 complex, locus D, wherein the contacting is performed in the presence or absence of a test compound; and b) measuring Th17 cell differentiation in the cell population in the presence or absence of the test compound, wherein an increase in Th17 cell differentiation in the cell population in the presence of the test compound relative to the absence of the test compound is a positive indicator that the test compound promotes Th17 differentiation.

In an embodiment of method for identifying a compound that promotes Th17 differentiation, the increase in the Th17 differentiation is detected as an increase in Th17 cell numbers, Th17 cell activity, or expression of Th17 specific cytokines. In a further embodiment, the increase in Th17 cell activity or expression of Th17 specific cytokines is detected as an increase in expression of at least one of RORγt, IL-17A, IL-17F, IL-22, IL23, IL23R, CD161, and CCR6. The method may be performed in a non-human animal or in vitro.

Also encompassed herein, is a composition comprising a single species of Th17 inducing bacteria or a component thereof, and a pharmaceutically acceptable buffer, for use in treating a patient with a pathogenic bacteria-related disorder, wherein said composition alleviates symptoms of the pathogenic bacteria-related disorder in the patient when administered to the patient in a therapeutically effective amount.

Use of a therapeutically effective amount of a composition comprising a single species of Th17 inducing bacteria or a component thereof, and a pharmaceutically acceptable buffer in the manufacture of a medicament for treating a patient with a pathogenic bacteria-related disorder, wherein the medicament alleviates or prevents symptoms of the pathogenic bacteria-related disorder when administered to the patient is also envisioned herein.

A method for treating a subject infected with a pathogenic bacteria is also described herein, the method comprising administering to the subject a therapeutically effective amount of a single species of Th17 inducing bacteria or a component thereof to the subject, wherein the administering alleviates or prevents symptoms of the pathogenic bacteria-related disorder, thereby treating the pathogenic bacteria-related disorder in the subject.

In a particular embodiment, the single species of Th17 inducing bacteria of the composition, use, or method is segmented filamentous bacteria (SFB). In alternative embodiments, the single species of Th17 inducing bacteria of the composition, use, or method is a homologue of SFB present in human microbiota, a human commensal species other than SFB, or a spore formant such as a Clostridia spp. or a Bacillus spp.

In a further embodiment, the composition, use, or method further comprises measuring Th17 cell differentiation in the subject wherein an increase in the Th17 cell differentiation in the subject after the administering relative to prior to the administering is determined. The increase in the Th17 cell differentiation in the subject may be determined using standard procedures known in the art, such as measuring Th17 cell differentiation in the subject prior to the administering and measuring Th17 cell differentiation in the subject after the administering and comparing the measurements before and after the administering to determine if there is a relative increase between the two measurements.

A composition comprising at least one SFB induced host cell molecule, and a pharmaceutically acceptable buffer, for use in treating a patient with a pathogenic bacteria-related disorder, wherein said composition alleviates symptoms of the pathogenic bacteria-related disorder in the patient when administered to the patient in a therapeutically effective amount is also encompassed herein.

Also encompassed herein, is a use of a therapeutically effective amount of a composition comprising at least one SFB induced host cell molecule, and a pharmaceutically acceptable buffer in the manufacture of a medicament for treating a patient with a pathogenic bacteria-related disorder, wherein the medicament alleviates or prevents symptoms of the pathogenic bacteria-related disorder when administered to the patient.

Also described herein, is a method for treating a subject infected with a pathogenic bacteria, the method comprising administering to the subject a therapeutically effective amount of at least one SFB induced host cell molecule in a pharmaceutically acceptable buffer to the subject, wherein the administering alleviates or prevents symptoms of the pathogenic bacteria-related disorder, thereby treating the pathogenic bacteria-related disorder in the subject.

In an embodiment, the at least one SFB induced host cell molecule of the composition, use, or method is serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; or lymphocyte antigen 6 complex, locus D.

In an embodiment, the at least one SFB induced host cell molecule of the composition, use, or method is at least one of serum amyloid A 1, resistin like beta, or serum amyloid A 2. In a still further embodiment, the at least one SFB induced host cell molecule is a combination of two of serum amyloid A 1, resistin like beta, or serum amyloid A 2 or a combination of serum amyloid A 1, resistin like beta, and serum amyloid A 2. In yet another embodiment, the at least one SFB induced host cell molecule is at least one of serum amyloid A 1 or serum amyloid A 2. In a further embodiment, the at least one SFB induced host cell molecule is a combination of serum amyloid A 1 and serum amyloid A 2.

Also encompassed herein are compositions, uses, and methods further comprising monitoring efficacy of the administering in the subject by monitoring Th17 cell differentiation in the subject. As described herein, monitoring Th17 cell differentiation may comprise measuring Th17 cell numbers, Th17 cell activity, or expression of Th17 specific cytokines Increases in Th17 cell activity or expression of Th17 specific cytokines may be detected as an increase in expression of at least one of RORγt, IL-17A, IL-17F, IL-22, IL23, IL23R, CD161, and CCR6. In an embodiment, Th17 cell differentiation is measured in a sample isolated from the patient or subject. Exemplary samples include a blood sample and a biopsy sample.

In another aspect, the administering of the composition, use, or method involves a patient infected with a pathogenic bacteria or at risk for infection with a pathogenic bacteria. In particular embodiment of the invention, the pathogenic bacteria is an antibiotic resistant pathogenic bacteria. In an embodiment thereof, the efficacy of the administering of the composition, use, or method involving a patient infected with a pathogenic bacteria is positively correlated with a reduction in the number of pathogenic bacteria in the infected patient. More particularly, efficacy can be evaluated by determining the number of pathogenic bacteria in an infected patient before and after the administering and comparing the two measurements of bacterial load to determine if the number of pathogenic bacteria is reduced following the administering, wherein a reduction following the administering is positively correlated with efficacy.

Efficacy may also be determined by comparing the number of pathogenic bacteria in a subject following exposure of the subject to the pathogenic bacteria, wherein the subject has been administered a composition, medicament, or received a treatment method as described herein, to the number of bacteria in a control subject following exposure of the control subject to the pathogenic bacteria alone, and comparing the number of pathogenic bacteria in the subject to the control subject, wherein a reduction in the number of pathogenic bacteria in the subject relative to that of the control subject is positively correlated with efficacy of the composition, medicament, or treatment method.

Other features and advantages of the invention will be apparent from the following description of the particular embodiments thereof, and from the claims.

### BRIEF DESCRIPTION OF THE DRAWINGS

FIG. 1. Comparative analysis of the microbiota in the terminal ileum of C57BL/6 (B6) mice from Jackson Laboratory versus Taconic Farms. A. Lumenal bacteria from both cecum and terminal ileum can induce Th17 cell differentiation upon transfer into Jackson B6 mice. Jackson B6 mice were gavaged with water (control) or with intestinal luminal contents from cecum or terminal ileum of Taconic B6 mice. LPL from small intestine were isolated 10 days later and analyzed for intracellular cytokines Representative plots from one experiment with 3 mice per group. Plots gated on TCRIβ⁺CD4⁺ LPL. B. Phylogenetic tree based on 16S rRNA gene sequences of bacterial taxa detected in the terminal ileum showing significantly different relative abundances (PhyloChip fluorescence intensity) between the suppliers, Taconic and Jackson. Branches of the tree are color coded according to phylum, while green and red bars display taxa with significantly greater relative abundance in Taconic and Jackson mice, respectively. The inner and outer dotted rings represent intensities corresponding to 5-fold and 25-fold differences in 16S copy number. The two taxa with the greatest difference between Taconic and Jackson mice, Lactobacillus murinus (˜94-fold difference) and Candidatus Arthromitus (˜40-fold difference), are noted by arrows.

FIG. 2. Segmented Filamentous Bacteria (SFB) in the intestinal tract of Th17 cell-sufficient and Th17 cell-deficient mice. A. Quantitative PCR (qPCR) analysis of SFB and total bacterial (EUB) 16S rRNA genes in mouse feces from Taconic (Tac) and Jackson (Jax) B6 mice. Genomic DNA was isolated from combined fecal pellets from 4 animals from each strain. The experiment was repeated numerous times with similar results. B. Scanning (SEM) and transmission (TEM) electron microscopy of terminal ileum of 8 week-old Jackson (Jax) and Taconic (Tac) C57BL/6 mice housed under similar conditions and diet for at least one week. Note the presence of long filamentous bacteria with SFB morphology in Taconic, but not Jackson mice. C. qPCR analysis for SFB presence in Jackson B6 mice after 14 days of co-housing with Taconic B6 mice (Jax-Coh). Genomic DNA was isolated from pooled feces from 3-4 mice per group. D. SFB colonization of terminal ileum of Jackson B6 mice after 14 days of co-housing with Taconic B6 mice (Jax-Coh). Toluidine-blue sections were prepared from 0.5 cm piece of the terminal ileum as described in Methods and examined by light microscopy. Adherent bacteria with SFB morphology were counted in 4-5 sections from each sample. Each column represents a separate animal.

FIG. 3. SFB specifically induce Th17 cell differentiation in germ-free mice. A-B. 6-week old Swiss-Webster (SW) germ-free mice (GF) were colonized with SFB (GF+SFB) as described in Methods and small intestinal lamina propria lymphocytes (SI LPL) were isolated 10 days later. Representative plots in (A) and combined data in (B) of IL-17 and IL-22 expression in TCRβ⁺CD4⁺ LPL. Data are from one of three separate experiments with similar results. Error bars (SD). SPF—mice raised under conventional specific pathogen free conditions. Each circle in (B) represents a separate animal. C-D. IL-17 (C) and IFNγ (D) expression in TCRβ⁺CD4⁺ SI LPL from mice colonized with different commensal bacteria. IQI germ-free (GF) mice were colonized with 16 strains of Bacteroidaceae (B. mix), SFB, 46 strains of Clostridium sp mixture (Clost. mix), or microbiota from conventionally raised mice (SPF). Intracellular cytokine production in SI LP CD4 T cells was analyzed 3 weeks later by flow cytometry. Circles represent separate animals. E. SFB colonization induces RORγt expression only in CD4⁺ T cells. RORγt expression in total SI LPL (top panels) and RORγt and Foxp3 expression in TCRβ⁺CD4⁺ SI LPL (bottom panels) in GF mice, GF mice colonized with SFB (GF+SFB) and conventionally raised mice (SPF).

FIG. 4. SFB induce Th17 cell differentiation upon colonization of Jackson C57BL/6 mice. A. Colonization of the terminal ileum by SFB 10 days after transfer of fecal homogenates from SFB-mono mice into Jackson B6 mice. B-C. IL-17 and IL-22 expression in TCRβ⁺CD4⁺ SI LPL in Jackson B6 mice colonized with SFB (Jax+SFB) compared to controls (Jax). Data from one of two experiments. D-E. Jackson microbiota induces Th17 cells only when complemented with SFB. Germ-free IQI mice were colonized with SFB, Jackson microbiota isolated from fecal pellets by itself (Jax), or a mixture of both (Jax+SFB). Th17 cell proportions were analyzed in the LP 3 weeks later by flow cytometry. Plots in (D) gated on total lymphocytes. Data in (E) represent percentage of IL-17⁺ cells in the CD4⁺ gate. F. RT-PCR for Th17 cell effector cytokines in total RNA from terminal ileum of the mice in (E)

FIG. 5. Transcriptional programs induced by SFB colonization. A. Venn diagrams showing the overlap between genes affected by either SFB colonization of SW GF mice only (Group 2), introduction of Taconic microbiota into Jackson B6 mice by co-housing (Group 1), or both (Group 3). Total RNA was prepared from terminal ileum of the corresponding mice after 10 days of colonization and Affymetrix gene chip analysis was performed as described in Methods. B. Heat-map analysis of the three groups in (A). Each line represents a single Affymetrix probe and each column a single mouse. Green, probes that were at least 2 fold down-regulated. Red, probes that were at least 2 fold up-regulated. C. Biological processes specifically induced by SFB (genes in Group 3 in (A)). Gene ontology analysis was performed as described in Methods. D. Changes in anti-microbial peptide related genes upon SFB colonization and Th17 cell induction by co-housing. Each column represents an individual mouse. E. RT-PCR analysis of selected genes, induced by SFB colonization. IQI GF mice (GF) were colonized with fecal homogenates from SFB-mono mice (SFB), Jackson B6 mice (Jackson), or a mixture of both (Jackson+SFB). Total RNA from terminal ileum was prepared 3 weeks later and RT-PCR performed as described in Methods. F. Top up-regulated genes in Group 3 in (A) arranged by fold change in GF+SFB mice.

FIG. 6. SFB colonization induces SAA expression that influences Th17 differentiation. A. Relative mRNA expression levels of SAA1-3 genes by real-time RT-PCR in the terminal ileum of IQI GF mice (GF) colonized with fecal homogenates from SFB-mono mice (SFB), Jackson B6 mice (Jackson), or a mixture of both (Jackson+SFB). B. Splenic naive CD4⁺ T cells were cocultured with or without LP CD11c⁺ cells in the presence of an anti-CD3 antibody with the indicated concentration of recombinant Apo-SAA for 4 days. T cells were collected, restimulated with PMA and ionomycin, and real-time RT-PCR performed. Results were normalized to expression of GAPDH mRNA. The data are representative of four independent experiments with similar results.

FIG. 7. SFB colonization confers protection from infection by Citrobacter rodentium. A. Jackson B6 mice (Jax) were co-housed with Taconic B6 mice (Jax CoH) for 10 days to induce colonization with SFB and Th17 cells. Both groups were infected with ˜1×10⁹ CFU of C. rodentium/mouse and pathogen colonization of the colon was examined at day 8 of infection (n=9/group). Uninf—uninfected controls. B-D. IQI GF mice colonized with Jackson microbiota (Jax) or Jax+SFB were orally infected with 2×10⁹ CFU of C. rodentium, and colons were harvested at day 8 of infection (n=5/group). C. rodentium CFUs in proximal colon (B), histopathology (C) and crypt length (D) in the distal colon (H&E). Data represent means±SD and circles represent separate animals. Similar effects of SFB on C. rodentium colonization were observed in a separate experiment with C.B17 mice colonized with ASF with and without SFB.

FIG. 8. Enhancement of Th17 differentiation by SAA. Splenic CD4+ T cells were purified from OT-II transgenic mice by MACS, and cocultured in 24-well plates at 2×10⁵ cells/well with lamina propria CD11c+ cells (1×10⁵ cells/well) in the presence of OVA peptide (OVA323-339) with or without recombinant human SAA (5 μg/ml, Peprotech, cat#300-13) for 4 days. T cells were restimulated with PMA and ionomycin for 3 hr, and examined for expression of CD4 and IL-17 by FACS. Numbers represent the percentage of IL-17+ cells among CD4+ T cells.

FIG. 9. SAA induces IL-6 and IL-23 expression from intestinal DCs. Small intestinal CD11c+ cells were plated in 24-well plates at 2×10⁵ cells/well, stimulated with 5 μg/ml recombinant Apo-SAA for the indicated times, and real-time RT-PCR performed. Results were normalized to expression of GAPDH mRNA. Cultures were performed in duplicate wells and data shown as the mean+/−SD.

### DETAILED DESCRIPTION OF THE INVENTION

The present inventors have determined that specific members of the commensal microbiota known as segmented filamentous bacteria (SFB), with the candidate name Arthromitus, are potent inducers of Th17 cells in the SI LP of mice. SFB, spore-forming gram-positive bacteria most closely related to the genus Clostridium, have been reported to colonize the intestines of numerous species, including humans (Davis and Savage, 1974; Klaasen et al., 1993a). They typically adhere tightly to epithelium in the ileum, where their abundance has been noted to correlate with reduced colonization and growth of pathogenic bacteria (Garland et al., 1982; Heczko et al., 2000). SFB were present in large numbers in conventionally raised B6 mice from Taconic Farms, but were undetectable in the same strain of mice obtained from the Jackson Laboratory. Introduction of SFB, but not other bacteria, into Th17 cell-deficient mouse models induced IL-17 and IL-22 expression in CD4⁺ T cells in the SI LP. Upon colonization, SFB induced a pro-inflammatory gene program that was similar to that induced in Jackson B6 mice co-housed with Taconic animals, suggesting that SFB are major modulators of immune responses in conventional mice. SFB colonization induced production of serum amyloid A (SAA) in the terminal ileum, and SAA acted on lamina propria dendritic cells (LP DCs) to promote Th17 cell differentiation in vitro. SFB colonization resulted in reduced growth of an intestinal pathogen, suggesting that intestinal commensal microbes can contribute to Th17 cell-mediated mucosal protection.

More particularly, infection with a pathogenic bacteria (e.g., Citrobacter rodentium) was attenuated by the presence of SFB in the GI tract. SFB induced pro-inflammatory gene programs are positively correlated with the ability of a subject to mount an effective immune response to such pathogenic bacteria. Th17 cell activity is, furthermore, positively correlated with the subject's ability to mount an effective immune response to the pathogenic bacteria. The present findings, furthermore, suggest that SFB and other single species shown to have Th17 inducing properties and components thereof can be used in combination to promote Th17 differentiation and activity in a subject or patient for therapeutic and/or prophylactic purposes. Such therapeutic and/or prophylactic purposes extend beyond the gastrointestinal tract to distal sites of the patient's body, wherein an increase in Th17 differentiation and activity would confer therapeutic and/or prophylactic benefit to the subject or patient.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which are placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More particularly, the preparation comprises at least 75% by weight, and most particularly 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning Such methods include, but are not limited to, viral transduction, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a protein antigen, in a host having a functioning immune system. Immune responses may be humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. The adaptive immune system and innate immune system are understood in the art to contribute to immune responses. The differential contribution of these immune systems is dependent on the particular circumstances eliciting the immune response. Generally speaking, the innate immune system comprises cells and mechanisms that defend the host from infection by other organisms in a non-specific manner. Accordingly, innate system cells recognize and respond to pathogens in a generic way. The adaptive immune system comprises highly specialized, systemic cells and processes that respond to pathogenic challenges. The adaptive immune system confers the ability to recognize pathogens with specificity and generate memory with regard to recognition of the specific pathogen, such that a stronger response is elicited in future encounters with the pathogen. The adaptive immune system, therefore, confers lasting immunity to the host. Immune responses may be measured both in in vitro and in various cellular or animal systems.

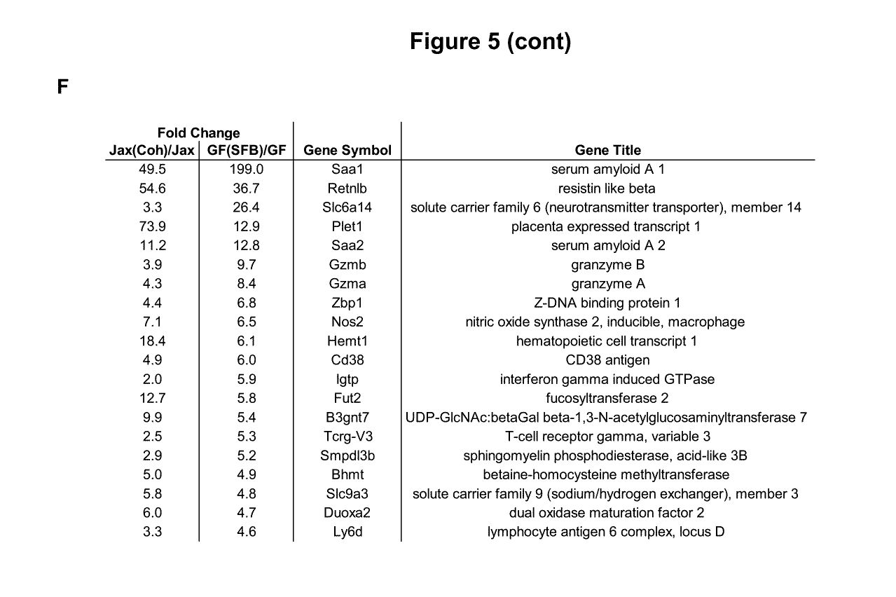

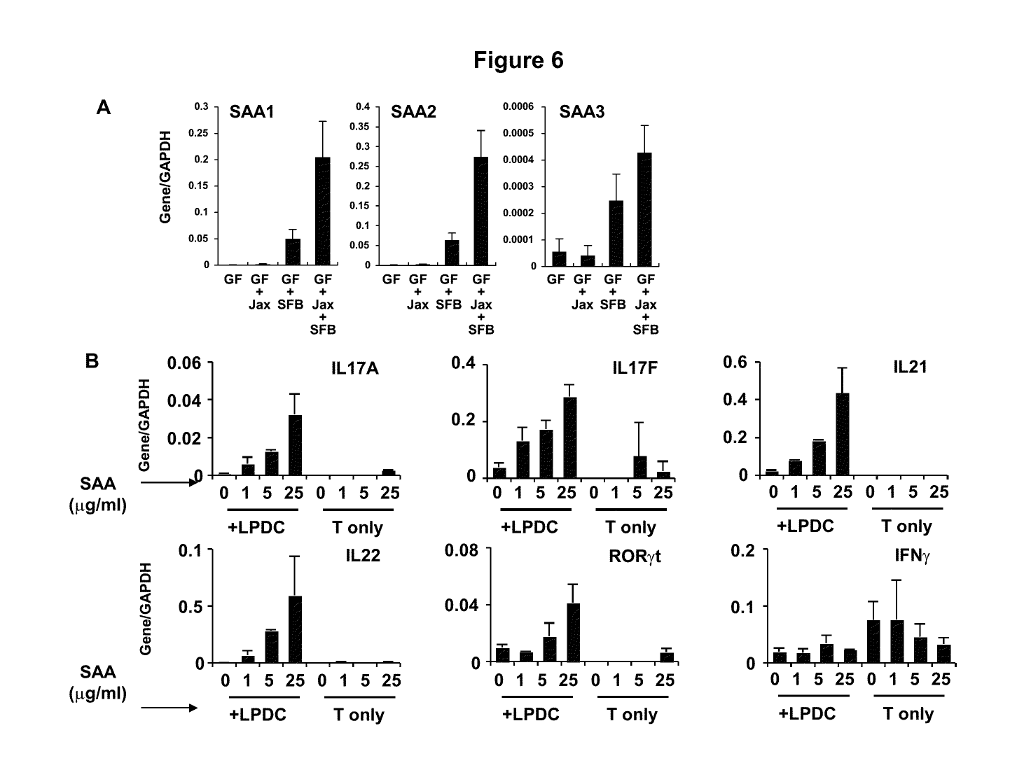

The methods and assays described herein and with which results depicted in FIGS. 6 and 7 were generated provide exemplary in vivo and in vitro systems for screening for compounds and/or synergistic combinations thereof that promote Th17 differentiation.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

As used herein, the term “bacterial species” refers to a homogeneous or highly similar bacterial population of the lowest or closest possible taxonomic identification or operational taxonomic unit (OTU). Whenever possible, similarity is assessed by examining similarity within highly variable regions of the 16S rRNA gene sequence. SFB, for example, are currently unclassified and are grouped in a provisional candidate genus designated Candidatus arthromitus. SFB are, however, distinguished by their morphology and relative conservation of their 16S rRNA gene sequence.

As used herein, the terms “pathogenic bacteria” is used to refer to bacteria that cause infectious disease. An exemplary list of human pathogenic bacteria includes the following genuses, and denotes exemplary species of each genus in parentheses: Bordetella (Bordetella pertussis); Borrelia (Borrelia burgdorferi); Brucella (Brucella abortus, Brucella canis, Brucella melitensi, Brucella suis); Campylobacter (Campylobacter jejuni); Chlamydia (Chlamydia pneumoniae, Chlamydia psittaci, Chlamydia trachomatis); Clostridium (Clostridium botulinum, Clostridium difficile, Clostridium perfringens, Clostridium tetani); Corynebacterium (Corynebacterium diphtheriae); Enterococcus (Enterococcus faecalis, Enterococcus faecium); Escherichia (Escherichia coli); Francisella (Francisella tularensis); Haemophilus (Haemophilus influenzae); Helicobacter (Helicobacter pylori); Legionella (Legionella pneumophila); Leptospira (Leptospira interrogans); Listeria (Listeria monocytogenes); Mycobacterium (Mycobacterium leprae, Mycobacterium tuberculosis); Mycoplasma (Mycoplasma pneumoniae); Neisseria (Neisseria gonorrhoeae, Neisseria meningitidis); Pseudomonas (Pseudomonas aeruginosa); Rickettsia (Rickettsia rickettsii); Salmonella (Salmonella typhi, Salmonella typhimurium); Staphylococcus (Staphylococcus aureus, Staphylococcus epidermidis, Staphylococcus saprophyticus); Streptococcus (Streptococcus agalactiae, Streptococcus pneumoniae, Streptococcus pyogenes); Treponema (Treponema pallidum); Vibrio (Vibrio cholerae); Yersinia (Yersinia pestis) and vancomycin-resistant Enterococci (VRE) Salmonella spp.

Enteric bacteria frequently associated with disease in humans include: Campylobacter jejuni, pathogenic strains of Escherichia coli (e.g., E. coli 0157), Salmonella species (e.g., S. typhosa, S. paratyphi, S. schottmuelleri), Shigella species (S. dysenteriae, S. flexneri), Vibrio cholerae, Proteus vulgaris, Pseudomonas aeruginosa, and Helicobacter pylori.

Pathogenic bacteria, such as those listed herein above, cause a variety of illnesses, many of which are fatal if untreated. Tuberculosis, for example, is caused by the bacterium Mycobacterium tuberculosis and kills about 2 million people a year. Pathogenic bacteria also contribute to other globally pervasive diseases, including pneumonia, which can be caused by Streptococcus and Pseudomonas bacteria, and illnesses contracted by eating contaminated food, which can be caused by Shigella, Campylobacter, and Salmonella. Pathogenic bacteria are also causative agents in tetanus, typhoid fever, diptheria, syphilis, and leprosy. Accordingly, Salmonellosis, VRE infection, Clostridium difficile-Associated Disease (CDAD), Multidrug-resistant Organism (MDRO) infections are pathogenic bacterial disease/disorder targets for the compositions, uses, and methods described herein.

SFB Induced Immune Response Program in the Gut

To identify specific effects of SFB, we compared the gene expression profiles in the terminal ileum of Swiss-Webster germ-free (GF) mice before and after colonization with SFB and in Jackson B6 mice before and after cohousing with Taconic B6 animals. Colonization of GF mice with SFB induced at least a 2-fold change in expression of 253 genes, while cohousing of Jackson B6 mice with Taconic B6 mice induced a similar change in 470 genes. More importantly, there was a high degree of overlap between the two groups, with expression of 131 genes affected by both treatments. Three groups of genetic profiles were thus distinguished. Group 1 includes genes whose expression was affected only in Jackson mice by cohousing, but was not statistically different after SFB colonization. This group most likely includes genes whose expression is influenced by microbiota other than SFB that differs between the mice from the different vendors, as well as strain-specific changes. Group 2 consists of genes whose expression only changed in GF mice upon colonization with SFB, but not in Jackson B6 mice following cohousing. A subset of these genes is expected to reflect changes induced in GF animals upon general intestinal colonization with bacteria. Group 3 includes the genes with expression differences after both SFB colonization and cohousing with Taconic mice and thus contains genes specifically induced by SFB and associated with Th17 cell induction.

SFB exerted an inductive effect in the host, which was demonstrated by the finding that most (>70%) of the genes in group 3 were upregulated after SFB colonization. By comparison, most genes in group 1 (>70%) were downregulated, which suggests that the rest of the Taconic microbiota has a suppressive effect that may possibly restrain the inductive effect of SFB. Group 2, on the other hand, consisted of roughly equal numbers of upregulated and downregulated genes.

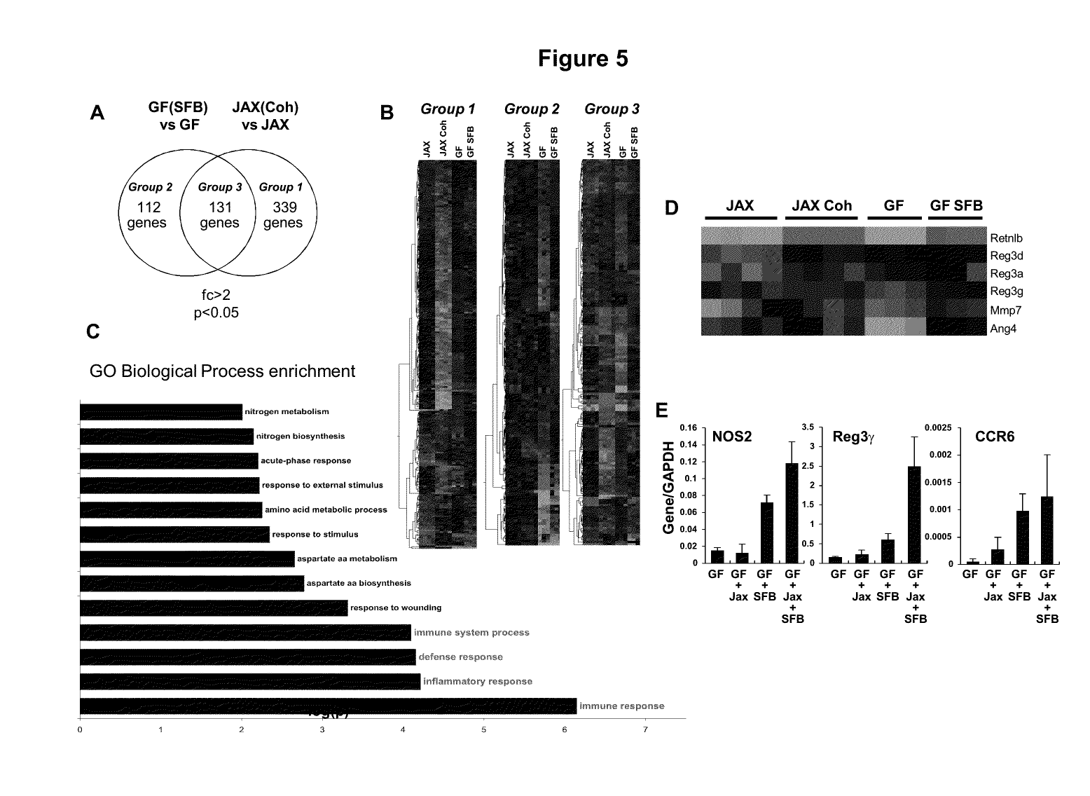

To evaluate changes specifically associated with Th17 cell-inducing SFB, we next concentrated on the genes in group 3. A list of the top upregulated genes is presented in FIG. 5F. A gene ontology (GO) biological pathway analysis of upregulated genes in group 3 showed that immune system pathways were among the programs most significantly induced by SFB and raised the possibility that at least some of the observed gene expression changes were mediated by Th17 cells or their effector cytokines. Because IL-17 and IL-22 have been associated with induction of antimicrobial peptides (AMPs) (Curtis and Way, 2009, Immunology 126:177-185; Kolls et al., 2008, Nat. Rev. Immunol. 8:829-835; Zheng et al., 2008, Nat. Med. 14:282-289), we compared the induction of AMP-related genes on arrays. Multiple AMP genes were induced specifically by colonization with SFB, consistent with an upregulated Th17 cell response. Upregulation of Th17 cell-associated genes (Il117, Il21, Ccr6, Nos2) and AMPs (Reg3g) after SFB colonization was confirmed by quantitative RT-PCR. See also Ivanov et al. (2009) Cell 139:485-498, the entire contents of which are incorporated herein in their entirety.

SFB Induced Host Cell Molecules

The top upregulated SFB induced host cell molecules, including serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; and lymphocyte antigen 6 complex, locus D can be synthesized using standard protocols known to skilled artisans or purchased. The SFB induced host cell molecules can be isolated from natural sources, expressed in a suitable cell-based expression system, synthesized from amino acid building blocks in vitro using protocols described herein and known to skilled artisans, or purchased from vendors. In exemplary fashion, serum amyloid A (SAA)-1 and -2 may be isolated from plasma as described, for example, in Kaplan et al. [Journal of Chromatography B 704:69 (1997); the entire contents of which is incorporated herein in its entirety], expressed using a suitable cell-based expression system, or purchased, for example, from PreproTech Inc. (Rocky Hill, N.J.). Mouse and human resistin like beta and murine granzyme B, are also available for purchase from PreproTech Inc.

With regard to synthesis of the SFB regulated (induced and repressed) host cell molecules identified herein using suitable cell-based expression systems, DNA constructs comprising nucleic acid sequences encoding such molecules can be designed to optimize for expression and purification of polypeptides encoded therefrom as described herein and known in the art. Nucleic acid and/or amino acid sequence information relating to SFB regulated (induced and repressed) host cell molecules is known and can be accessed via publicly available databases using the accession numbers as indicated below.

SFB induced host cell molecules (host cell molecules whose expression is specifically induced by SFB by 2-fold or greater) and SFB repressed host cell molecules (host cell molecules whose expression is specifically reduced by SFB by 2-fold or greater) include the following host cell molecules, which are further identified with relevant accession numbers for Unigene and Entrez Gene databases.

Upregulated Genes Specifically Induced by SFB:

| Unigene | Gene | Entrez |
|---|---|---|
| (Avadis) | Symbol | Gene |
| Mm.148800 | Saa1 | 20208 |
| Mm.24045 | Rsad2 | 58185 |
| Mm.878 | Ly6d | 17068 |
| Mm.33902 | Igtp | 16145 |
| Mm.21123 | Retnlb | 57263 |
| Mm.29959 | 1600029D21Rik | 76509 |
| Mm.200941 | Saa2 | 20209 |
| Mm.261564 | Slc9a3 | 105243 |
| Mm.462929 | Ak3l1 * | 100047616 |
| Mm.180191 | Psmb8 | 16913 |
| Mm.86467 | B3gnt7 | 227327 |
| Mm.290046 | Fut2 | 14344 |
| Mm.364155 | Tcrg-V3 | 21637 |
| Mm.422756 | Il18bp | 16068 |
| Mm.14874 | Gzmb | 14939 |
| Mm.14277 | Saa3 | 20210 |
| Mm.284248 | Ccl5 | 20304 |
| Mm.2893 | Nos2 | 18126 |
| Mm.133083 | Prss27 * | 213171 |
| Mm.228363 | Oasl2 | 23962 |
| Mm.297393 | Herc5 | 67138 |
| Mm.42029 | Ccl8 * | 100048554 |
| Mm.15510 | Gzma | 14938 |
| Mm.143804 | Myot | 58916 |
| Mm.436843 | Cyp2d34 | 223706 |
| Mm.4497 | Ptk6 | 20459 |
| Mm.23347 | Pla2g5 | 18784 |
| Mm.329582 | Bhmt | 12116 |
| | Al451557 | 102084 |
| Mm.171333 | Duoxa2 | 66811 |
| Mm.116687 | Zbp1 | 58203 |
| Mm.31852 | Tifa * | 637082 |
| Mm.255848 | Hk2 | 15277 |
| Mm.283573 | Gsr | 14782 |
| Mm.390785 | Gml /// Hemt1 | 15202 /// 625599 |
| Mm.4610 | Upp1 | 22271 |
| Mm.212991 | 2310056P07Rik | 70186 |
| | LOC630729 | 630729 |
| Mm.402 | Zc3h12a | 230738 |
| Mm.17185 | Lgmn | 19141 |
| Mm.3468 | Socs3 | 12702 |
| Mm.295284 | Stom | 13830 |
| Mm.348025 | Lrg1 | 76905 |
| Mm.311629 | Unc5cl | 76589 |
| Mm.1410 | Il18 | 16173 |
| Mm.766 | Cxcl9 | 17329 |
| Mm.19669 | Pfkfb3 | 170768 |
| Mm.253984 | Slc6a14 | 56774 |
| Mm.312628 | Serpina3g | 20715 |
| Mm.154783 | B3galt5 | 93961 |
| Mm.249873 | Cd38 | 12494 |
| Mm.261140 | Iigp1 * | 100044196 |
| Mm.247272 | Nfkbiz | 80859 |
| Mm.195060 | H2-DMb1 /// | 14999 /// |
| | H2-DMb2 | 15000 |
| Mm.28756 | Slc40a1 | 53945 |
| Mm.257931 | Rassf4 | 213391 |
| Mm.85429 | Steap1 | 70358 |
| Mm.4825 | Mmp7 | 17393 |
| Mm.330731 | Tgm2 | 21817 |
| Mm.226708 | Cyp2d9 | 13105 |
| Mm.41779 | Rabl5 | 67286 |
| Mm.347407 | Cebpd | 12609 |
| Mm.28110 | Tat | 234724 |
| Mm.452174 | Tgtp * | 620913 |
| Mm.313181 | Gna14 | 14675 |
| Mm.2942 | Asns | 27053 |
| | LOC665506 | 665506 |
| Mm.4138 | Dmbt1 | 12945 |
| Mm.57225 | Gpx2 | 14776 |
| Mm.20897 | Clca2 | 80797 |
| Mm.29981 | Bhmt2 | 64918 |
| Mm.45481 | 1110032A04Rik | 66183 |
| Mm.287187 | Smpdl3b | 100340 |
| Mm.22680 | Celsr1 | 12614 |
| | Ighg | 380794 |
| Mm.30300 | Ceacam10 | 26366 |
| Mm.392 | Indo | 15930 |
| Mm.39040 | Mal | 17153 |
| Mm.45054 | Gda | 14544 |
| Mm.326349 | Igl-J2 /// Igl-V1 | 16142 /// |
| | | 404739 |
| Mm.290600 | Clca4 | 229927 |
| Mm.38192 | Slfn4 | 20558 |

* Probe sets that align to several loci have been abbreviated in the table. The full listings are below:

| | |
|---|---|
| Ak3l1 /// LOC100047616 /// LOC635960 | 100047616 /// 11639 /// 635960 |
| LOC100048538 /// Prss27 | 100048538 /// 213171 |
| Ccl8 /// LOC100048554 | 100048554 /// 20307 |
| LOC637082 /// Tifa | 211550 /// 637082 |
| Iigp1 /// LOC100044196 | 100044196 /// 60440 |
| OTTMUSG00000005523 /// Tgtp | 21822 /// 620913 |

Downregulated Genes Specifically Induced by SFB:

| Unigene | Gene | Entrez |
|---|---|---|
| (Avadis) | Symbol | Gene |
| Mm.27227 | 5730469M10Rik | 70564 |
| Mm.155678 | Slc5a4b | 64454 |
| Mm.79983 | Plod2 | 26432 |
| Mm.190508 | Agr3 | 403205 |
| Mm.214923 | BC089597 | 216454 |
| Mm.8728 | Aqp7 | 11832 |
| Mm.33921 | Lrat | 79235 |
| Mm.472860 | Spsb4 | 211949 |
| Mm.473754 | Maob | 109731 |
| Mm.200307 | Slc45a3 | 212980 |
| Mm.3506 | Arg2 | 11847 |
| Mm.147226 | Mt2 | 17750 |
| Mm.6696 | Rdh7 | 54150 |
| Mm.423078 | Fbp1 | 14121 |
| Mm.154797 | Slc5a4a | 64452 |
| Mm.34365 | Dusp12 | 80915 |
| Mm.250719 | Bche | 12038 |
| Mm.273838 | Calcb | 116903 |
| Mm.41963 | Slc6a20b | 22599 |
| Mm.5323 | Slc25a45 | 107375 |
| Mm.334199 | Acsm3 | 20216 |
| Mm.14089 | Cyp1a1 | 13076 |
| Mm.46306 | Reg4 | 67709 |
| Mm.259074 | LOC100044232 /// | 100044232 /// |
| | Rasgef1b | 320292 |
| Mm.440465 | Gls | 14660 |
| Mm.100741 | Slc47a1 | 67473 |
| Mm.196189 | Angptl4 | 57875 |
| Mm.179195 | 1700057G04Rik | 78459 |
| Mm.46182 | Cda | 72269 |
| Mm.53865 | Ankrd29 | 225187 |
| Mm.52526 | 2810439F02Rik | 72747 |
| Mm.25311 | 1810015C04Rik | 66270 |
| Mm.39738 | Pcdh19 | 279653 |
| Mm.23575 | Gprc5a | 232431 |
| | Hsd3b3 | 15494 |
| Mm.55289 | Plscr4 | 235527 |
| Mm.205266 | Acaa1a /// Acaa1b | 113868 /// 235674 |
| Mm.2436 | Bhlhb2 | 20893 |
| Mm.207354 | Abcb1a | 18671 |
| Mm.24547 | Aadac | 67758 |
| Mm.158717 | Hsd3b2 /// Hsd3b3 /// | 15493 /// |
| | Hsd3b6 | 15494 /// 15497 |
| Mm.6979 | Trdmt1 | 13434 |
| Mm.332844 | Cyp3a11 | 13112 |
| Mm.439693 | Serpina1a /// | 20700 /// 20701 |
| | Serpina1b | |
| Mm.439693 | Serpina1b | 20701 |
| Mm.385180 | Fmo5 /// | 100046051 /// |
| | LOC100046051 | 14263 |
| Mm.271190 | Cyp2c65 | 72303 |
| Mm.142581 | Cyp2c55 | 72082 |

List of Gene Names of Genes Upregulated by SFB (82 genes): serum amyloid A 1; radical S-adenosyl methionine domain containing 2; lymphocyte antigen 6 complex, locus D; interferon gamma induced GTPase; resistin like beta; RIKEN cDNA 1600029D21 gene; serum amyloid A 2; solute carrier family 9 (sodium/hydrogen exchanger), member 3; adenylate kinase 3 alpha-like 1 ///similar to adenylate kinase 4; proteasome (prosome, macropain) subunit, beta type 8 (large multifunctional peptidase 7); UDP-GlcNAc:betaGal beta-1,3-N-acetylglucosaminyltransferase 7; fucosyltransferase 2; T-cell receptor gamma, variable 3; interleukin 18 binding protein; granzyme B; serum amyloid A 3; chemokine (C-C motif) ligand 5; nitric oxide synthase 2, inducible, macrophage; protease, serine 27 /// similar to protease, serine 27; 2′-5′ oligoadenylate synthetase-like 2; hect domain and RLD 5; chemokine (C-C motif) ligand 8 /// similar to monocyte chemoattractant protein-2 (MCP-2); granzyme A; myotilin; cytochrome P450, family 2, subfamily d, polypeptide 34; PTK6 protein tyrosine kinase 6; phospholipase A2, group V; betaine-homocysteine methyltransferase; expressed sequence AI451557; dual oxidase maturation factor 2; Z-DNA binding protein 1; TRAF-interacting protein with forkhead-associated domain /// similar to Traf2 binding protein; hexokinase 2; glutathione reductase; hematopoietic cell transcript 1 /// GPI anchored molecule like protein; uridine phosphorylase 1; RIKEN cDNA 2310056P07 gene; similar to Glutathione reductase, mitochondrial precursor (GR) (GRase); zinc finger CCCH type containing 12A; legumain; suppressor of cytokine signaling 3; stomatin; leucine-rich alpha-2-glycoprotein 1; unc-5 homolog C (C. elegans)-like; interleukin 18; chemokine (C—X—C motif) ligand 9; 6-phosphofructo-2-kinase/fructose-2,6-biphosphatase 3; solute carrier family 6 (neurotransmitter transporter), member 14; serine (or cysteine) peptidase inhibitor, clade A, member 3G; UDP-Gal:betaGlcNAc beta 1,3-galactosyltransferase, polypeptide 5; CD38 antigen; interferon inducible GTPase 1 /// hypothetical protein LOC100044196; nuclear factor of kappa light polypeptide gene enhancer in B-cells inhibitor, zeta; histocompatibility 2, class II, locus Mb1 /// histocompatibility 2, class II, locus Mb2; solute carrier family 40 (iron-regulated transporter), member 1; Ras association (RalGDS/AF-6) domain family member 4; six transmembrane epithelial antigen of the prostate 1; matrix metallopeptidase 7; transglutaminase 2, C polypeptide; cytochrome P450, family 2, subfamily d, polypeptide 9; RAB, member of RAS oncogene family-like 5; CCAAT/enhancer binding protein (C/EBP), delta; tyrosine aminotransferase; T-cell specific GTPase /// predicted gene, OTTMUSG00000005523; guanine nucleotide binding protein, alpha 14; asparagine synthetase; similar to T-cell receptor beta-2 chain C region; deleted in malignant brain tumors 1; glutathione peroxidase 2; chloride channel calcium activated 2; betaine-homocysteine methyltransferase 2; RIKEN cDNA 1110032A04 gene; sphingomyelin phosphodiesterase, acid-like 3B; cadherin, EGF LAG seven-pass G-type receptor 1 (flamingo homolog, Drosophila); Immunoglobulin heavy chain (gamma polypeptide); CEA-related cell adhesion molecule 10; indoleamine-pyrrole 2,3 dioxygenase; myelin and lymphocyte protein, T-cell differentiation protein; guanine deaminase; immunoglobulin lambda chain, variable 1 /// immunoglobulin lambda chain, joining region 2; chloride channel calcium activated 4; and schlafen 4.

In a particular embodiment, resistin like beta is the at least one SFB induced host cell molecule included in any and all combinations of SFB induced host cell molecules used in the claimed compositions, uses, and methods.

In a particular embodiment, matrix metallopeptidase 7 and fucosyltransferase 2 are excluded from use in the claimed compositions, uses, and methods. Accordingly, the compositions, uses, and methods as claimed may include the proviso that matrix metallopeptidase 7 and fucosyltransferase 2 are not included or are excluded therefrom.

List of Gene Names of Genes Downregulated by SFB (48 genes): RIKEN cDNA 5730469M10 gene; solute carrier family 5 (neutral amino acid transporters, system A), member 4b; procollagen lysine, 2-oxoglutarate 5-dioxygenase 2; anterior gradient homolog 3 (Xenopus laevis); cDNA sequence BC089597; aquaporin 7; lecithin-retinol acyltransferase (phosphatidylcholine-retinol-O-acyltransferase); splA/ryanodine receptor domain and SOCS box containing 4; monoamine oxidase B; solute carrier family 45, member 3; arginase type II; metallothionein 2; retinol dehydrogenase 7; fructose bisphosphatase 1; solute carrier family 5, member 4a; dual specificity phosphatase 12; butyrylcholinesterase; calcitonin-related polypeptide, beta; solute carrier family 6 (neurotransmitter transporter), member 20B; solute carrier family 25, member 45; acyl-CoA synthetase medium-chain family member 3; cytochrome P450, family 1, subfamily a, polypeptide 1; regenerating islet-derived family, member 4; RasGEF domain family, member 1B /// hypothetical protein LOC100044232; glutaminase; solute carrier family 47, member 1; angiopoietin-like 4; RIKEN cDNA 1700057G04 gene; cytidine deaminase; ankyrin repeat domain 29; RIKEN cDNA 2810439F02 gene; RIKEN cDNA 1810015C04 gene; protocadherin 19; G protein-coupled receptor, family C, group 5, member A; hydroxy-delta-5-steroid dehydrogenase, 3 beta- and steroid delta-isomerase 3; phospholipid scramblase 4; acetyl-Coenzyme A acyltransferase 1A /// acetyl-Coenzyme A acyltransferase 1B; basic helix-loop-helix domain containing, class B2; ATP-binding cassette, sub-family B (MDR/TAP), member 1A; arylacetamide deacetylase (esterase); hydroxy-delta-5-steroid dehydrogenase, 3 beta- and steroid delta-isomerase 2 /// hydroxy-delta-5-steroid dehydrogenase, 3 beta- and steroid delta-isomerase 3 /// hydroxy-delta-5-steroid dehydrogenase, 3 beta- and steroid delta-isomerase 6; tRNA aspartic acid methyltransferase 1; cytochrome P450, family 3, subfamily a, polypeptide 11; serine (or cysteine) peptidase inhibitor, clade A, member 1a /// serine (or cysteine) preptidase inhibitor, clade A, member 1b; serine (or cysteine) preptidase inhibitor, clade A, member 1b; flavin containing monooxygenase 5 /// similar to Flavin containing monooxygenase 5; cytochrome P450, family 2, subfamily c, polypeptide 65; and cytochrome P450, family 2, subfamily c, polypeptide 55.

Accordingly, agents that inhibit the expression and/or activity of at least one of the above SFB downregulated genes may be used in the compositions, uses, and methods to treat or ameliorate symptoms associated with a pathogenic bacterial disorder as described herein.

In a particular embodiment, an agent that inhibits the expression and/or activity of aquaporin 7 is included in any and all combinations of inhibitors of SFB downregulated host cell molecules used in the compositions, uses, and methods to treat or ameliorate symptoms associated with a pathogenic bacterial disorder as described herein.

Agents

As used herein, an “agent”, “candidate compound”, or “test compound” may be used to refer to, for example, nucleic acids (e.g., DNA and RNA), carbohydrates, lipids, proteins, peptides, peptidomimetics, small molecules and other drugs.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

The term “control substance”, “control agent”, or “control compound” as used herein refers a molecule that is inert or has no activity relating to an ability to modulate a biological activity. With respect to the present invention, such control substances are inert with respect to an ability to modulate differentiation and/or activity, for example, of Th17 cells. Exemplary controls include, but are not limited to, solutions comprising physiological salt concentrations.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology, John Wiley & Sons, New York).

Agents Identified by the Methods of the Invention

The invention provides methods for identifying agents (e.g., candidate compounds or test compounds) capable of promoting pathways due to or triggered by commensal bacteria in the GI-tract that lead to Th17 differentiation and accumulation. Agents that are capable of promoting such pathways, as identified by methods of the invention, are useful as candidate anti-pathogenic bacteria therapeutics.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

Therapeutic Uses of Agents Identified

The invention provides for treatment of diseases or conditions due to the presence of pathogenic bacteria on mucosal surfaces (e.g., of the GI tract, lung, mouth, and nasal passages) and in proximal and distal non-mucosal sites by administration of a therapeutic agent identified using the above-described methods. Such agents include, but are not limited to proteins, peptides, protein or peptide derivatives or analogs, antibodies, nucleic acids, and small molecules.

The invention provides methods for treating patients afflicted with a disease caused by a pathogenic bacteria, for example, comprising administering to a subject an effective amount of a compound identified by the method of the invention. In a particular aspect, the compound is substantially purified (e.g., substantially free from substances that limit its effect or produce undesired side-effects). The subject is particularly an animal, including but not limited to animals such as cows, pigs, horses, chickens, cats, dogs, etc., and is more particularly a mammal, and most particularly a human. In a specific embodiment, a non-human mammal is the subject.

A list of diseases caused by pathogenic bacteria include without limitation: tuberculosis, pneumonia, tetanus, typhoid fever, diptheria, syphilis, leprosy, gonorrhea, diarrhea, ear infections, dysentery, septicemia, toxinoses, Rocky Mountain spotted fever, and botulism.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, e.g., an inflammatory site, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

Pharmaceutical Compositions

The present invention also provides pharmaceutical compositions. Such compositions comprise a therapeutically effective amount of an agent and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of a disease correlated with or caused by infection with pathogenic bacteria, for example, can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances.

As a further aspect of the invention the present compounds (e.g., SFB induced host cell molecules and bacterial components) are provided for use as a pharmaceutical especially in the treatment or prevention of the aforementioned conditions and diseases. Also provided herein are compositions comprising at least one of the present compounds and the use of the present compounds in the manufacture of a medicament for the treatment or prevention of one of the aforementioned conditions and diseases.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For treatment of long-term conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses.

The compounds of this invention can be administered as the sole active agent or they can be administered in combination with other agents, including other compounds that demonstrate the same or a similar therapeutic activity and that are determined to safe and efficacious for such combined administration.

Depending on the compound administered and the condition of the patient, and further to the above guidance, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

The dosage or dosing regime of an adult patient may be proportionally adjusted for children and infants, and also adjusted for other administration or other formats, in proportion for example to molecular weight or immune response. Administration or treatments may be repeated at appropriate intervals, at the discretion of the physician.

Nucleic Acids

The invention provides methods for identifying agents capable of promoting pathways triggered by commensal bacteria of the GI tract that lead to Th17 differentiation and/or accumulation. The invention further provides methods for identifying agents capable of promoting such pathways. Accordingly, the invention encompasses administration of a nucleic acid encoding a peptide or protein capable of promoting such pathways, as well as antisense sequences or catalytic RNAs capable of promoting these pathways.

Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein capable of promoting pathways triggered by commensal bacteria of the GI tract (e.g., SFB) the presence of which leads to Th17 differentiation and/or accumulation, such nucleic acid being part of an expression vector that expresses the peptide or protein in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA coding for a peptide or protein capable of promoting pathways triggered by commensal bacteria of the GI tract that lead to Th17 differentiation and/or accumulation may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

Unless defined otherwise, all technical and scientific terms used herein have the same meaning as commonly understood by one of ordinary skill in the art to which this invention belongs.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting, since the scope of the present invention will be limited only the appended claims.

### EXAMPLES

Methods and Materials

### Mice and Bacterial Strains

B6 mice were obtained from Taconic Farms or Jackson Laboratory. Swiss-Webster germ-free and conventionally raised (SPF) mice were purchased from Taconic Farms. Germ-free IQI mice were purchased from Japan CLEA Inc. Mice mono-colonized with SFB or 46 strains of Clostridia were developed previously (Itoh and Mitsuoka, 1985; Umesaki et al., 1995). To generate Bacteroides-associated mice, 16 strains of Bacteroides (six strains of B. vulgatus, seven of B. acidifaciens group 1, and three of B. acidifaciens group 2), which were originally isolated from murine intestinal commensal bacteria (Miyamoto and Itoh, 2000), were cultured on Eggerth-Gangon agar (Nissui) in an anaerobic stainless steel jar and inoculated orally into germ-free IQI mice.

PhyloChip Analysis

Six week old Jackson B6 and Taconic B6 mice were purchased from the corresponding vendor and housed for 3 weeks in separate microisolator cages at the NYUSOM animal facility to equilibrate housing conditions, including bedding and diet. Sample collection, processing and PhyloChip analysis are described in detail below.

PhyloChip Methods

Nucleic Acid Extraction from Mouse Small Intestines.

Distal halves of the small intestine from 4 mice from each group were dissected, flash-frozen liquid nitrogen, and stored at −80° C. (4 Jackson and 4 Taconic) were flash frozen in liquid nitrogen and stored at −80° C. For extraction, each frozen gut section was transferred with sterile forceps to a 2 mL Lysing Matrix E tube (MP Biomedicals) containing 50 μL of 0.1M aluminum ammonium sulfate. Equal volumes (500 μL) of modified CTAB buffer (10% CTAB, 250 mM phosphate, 300 mM NaCl) and phenol: chloroform:isoamylalcohol (25:24:1) were added to each tube, tubes were agitated with a FastPrep (MP Biomedicals: 30 seconds, 5.5 m/s) and centrifuged (5 min, 4° C., 16,000×g). Aqueous phases were removed by pipette, transferred to phase-lock gel tubes (MaXtract High Density Gel Tubes, Qiagen) containing approximately 1 aqueous volume of chloroform, inverted by hand and centrifuged again to yield a crude nucleic acid extract. Another 500 μL aliquot of modified CTAB buffer was added to the Lysing Matrix E tube and tubes were agitated and centrifuged again as described above. This second aqueous extract was purified with chloroform as above to yield an additional crude nucleic extract. This re-extraction step was repeated twice. Each of the three crude nucleic acid extracts from a sample was transferred from the phase-lock gel tube to an individual 2 mL tube, gently mixed by pipette with 2 volumes of polyetheleneglycol/salt solution (30% wt/vol PEG 6000, 1.6M NaCl), and incubated at room temperature for 2 hours. Crude nucleic acid extracts were then centrifuged (10 min, 4° C., 16,000×g), supernatants were removed by pipette and pellets were washed with 1 mL ice-cold 70% ethanol. Pellets were resuspended in 60 μL of nuclease-free (DEPC treated) water. The three crude nucleic acid extracts from each sample were combined and a 60 μL, aliquot was purified by column chromatography using an DNA/RNA Allprep Kit (Qiagen, Calif.). DNA and RNA were separately eluted and purified. Purified DNA was eluted in 2×25 μL, Buffer EB and used as genomic DNA template for PCR amplification.

PCR Amplification of 16S rRNA Genes.

Genomic DNA from the eight samples was quantified by NanoDrop 1000, and diluted in nuclease-free water to achieve a standard concentration of 500 ng/μL. Eight replicate polymerase chain reactions were prepared for each gut sample containing final concentrations of 10 ng/μL gDNA template, 0.02 U/μL ExTaq (Takara Bio Inc.,), 1× ExTaq buffer, 0.2 mM dNTP mixture, 1 μg/μL Bovine Serum Albumin (BSA), and 300 pM each of universal bacterial primers: 27F (5′-AGAGTTTGATCCTGGCTCAG-3′; SEQ ID NO: 1) and 1492R (5′-GGTTACCTTGTTACGACTT-3′; SEQ ID NO: 2). To minimize PCR bias due to variable template annealing efficiencies and random effects PCR was performed on a BioRad iCycler with an eight temperature annealing gradient (48-58° C.) and the following conditions: 95° C. (3 min), followed by 30 cycles of 95° C. (30 sec), annealing (30 sec), 72° C. (2 min), and a final extension at 72° C. (10 min). Reactions were combined for each sample and concentrated with 0.8 volumes isopropanol, washed twice with ice cold 70% ethanol and resuspended in 50 μL, nuclease-free water.

PhyloChip Microarray Analysis of 16S rRNA Gene Diversity.

500 ng of pooled PCR amplicons of each sample were spiked with known concentrations of amplicons derived from yeast and bacterial metabolic genes. This mix was fragmented to 50-200 bp using DNase I (0.02 U μg₋₁ DNA, Invitrogen, Carlsbad, Calif., USA) and One-Phor-All buffer (GE Healthcare, Piscataway, N.J., USA) following the manufacturer's protocols. The mixture was then incubated at 25° C. for 20 min and 98° C. for 10 min before biotin labeling with a GeneChip DNA labeling reagent kit (Affymetrix, Santa Clara, Calif., USA) following the manufacturer's instructions. Next, the labeled DNA was denatured at 99° C. for 5 min and hybridized to custom-made Affymetrix GeneChips (16S rRNA genes PhyloChips) at 48° C. and 60 rpm for 16 h. PhyloChip washing and staining were performed according to the standard Affymetrix protocols described previously (Masuda and Church 2002).

Each PhyloChip was scanned and recorded as a pixel image, and initial data acquisition and intensity determination were performed using standard Affymetrix software (GeneChip microarray analysis suite, version 5.1). Background subtraction, data normalization and probe pair scoring were performed as reported previously (DeSantis, Hugenholtz et al. 2006; Brodie, Desantis et al. 2007; DeSantis, Brodie et al. 2007). The positive fraction (PosFrac) was calculated for each probe set as the number of positive probe pairs divided by the total number of probe pairs in a probe set. Taxa were deemed present when the PosFrac value exceeded 0.90. Intensities were summarized for each taxon/probe-set using a trimmed average (highest and lowest values removed before averaging) of the intensities of the perfect match probes (PM) minus their corresponding mismatch probes (PM).

Fecal DNA Extraction for qPCR

Bacterial genomic DNA was extracted from fresh or frozen fecal samples (within an experiment the samples were treated identically) by phenol-chloroform extraction. Briefly ˜100 mg of fecal sample was suspended in a solution containing 500 μl of DNA extraction buffer, 210 μl of 20% SDS, 500 μl of a mixture of of phenol:chloroform:isoamyl alcohol (25:24:1), and 500 μl of a slurry of 0.1-mm-diameter zirconia/silica beads (BioSpec Products, Bartlesville, Okla.). Cells were lysed by mechanical disruption with a FastPrep bead beater set on high for 45 sec, after which three rounds of phenol:chloroform extraction were performed. DNA was resuspended in TE buffer with 100 μg/ml RNase.

16S rRNA Gene Quantitative PCR Analysis

Bacterial genomic DNA was isolated from fecal pellets as described above. Quantitative PCR analysis was carried out as described in (Barman et al., 2008). Primer sequences for SFB and bacterial 16S rRNA genes as well as PCR conditions were as described in (Barman et al., 2008). For SFB, relative quantity was calculated by the ΔCt method and normalized by the presence of total bacteria (EUB primers), dilution and weight of the sample and presented as relative fold change to an external sample. Typical Ct values for SFB were ˜20 cycles and for EUB ˜11 cycles. Samples that were negative after 40 cycles were considered “not detected” (n.d.).

Gene Expression Analysis

RNA was prepared from terminal ileum as described (Ivanov et al., 2008). For microarray analysis, RNA was labeled and hybridized to GeneChip Mouse Genome 430 2.0 arrays following the Affymetrix protocols. Data were analyzed in GeneSpring GX10. Significant genes were selected based on p values smaller than 0.05 and fold change greater than 2. For enrichment analysis of biological process ontology, probe lists were analyzed in DAVID (Dennis et al., 2003; Huang da et al., 2009) and processes were selected based on p values smaller than 0.01.

Real-Time RT-PCR (Q-PCR).

cDNAs were synthesized from RNA samples prepared with a RNeasy Mini Kit (QIAGEN) using M-MLV Reverse Transcriptase (Promega). Real-time RT-PCR was performed using the ABI 7300 real time PCR system. Serial dilutions of a standard were included for each gene to generate a standard curve and allow calculation of the input amount of cDNA for each gene. Values were then normalized by the amount of GAPDH in each sample. Primer sequences are reported below.

RT-PCR Primer Sequences

Primers for IL-17A, IL-17F, IL-21, IL-22, RORγt and GAPDH have been described previously (Atarashi, Nishimura et al. 2008). The following primer sets were also used:

SAA1,
(SEQ ID NO: 3)
5′-CATTTGTTCACGAGGCTTTCC-3′
and
(SEQ ID NO: 4)
5′-GTTTTTCCAGTTAGCTTCCTTCATGT-3′;
SAA2,
(SEQ ID NO: 5)
5′-TGTGTATCCCACAAGGTTTCAGA-3′
and
(SEQ ID NO: 6)
5′-TTATTACCCTCTCCTCCTCAAGCA-3′;
SAA3,
(SEQ ID NO: 7)
5′-CGCAGCACGAGCAGGAT-3′
and
(SEQ ID NO: 8)
5′-CCAGGATCAAGATGCAAAGAATG-3′;
NOS2,
(SEQ ID NO: 9)
5′-TGCCCCTTCAATGGTTGGT-3′
and
(SEQ ID NO: 10)
5′-TCCTTCGGCCCACTTCCT-3′;
Reg3g,
(SEQ ID NO: 11)
5′-CCTTCCTCTTCCTCAGGCAAT-3′
and
(SEQ ID NO: 12)
5′-TAATTCTCTCTCCACTTCAGAAATCCT-3′;
MMP7,
(SEQ ID NO: 13)
5′-ATCAGTGGGAACAGGCTCAGA-3′
and
(SEQ ID NO: 14)
5′-TTGTCCACTAGACTATTGACCTTCTTTG-3′;
and
CCR6,
(SEQ ID NO: 15)
5′-TTGGTTCGCCACTCTAATCAGTAG-3′
and
(SEQ ID NO: 16)
5′-GCAGTTCAGCCACACTCTCACT-3′.

Co-Housing and Microbiota Reconstitution

Co-housing and microbiota reconstitutions were performed as described before (Ivanov et al., 2008). For inoculation of germ-free mice with SFB, fecal pellets were collected from SFB-monocolonized mice using sterilized test tubes in the vinyl-isolator and were preserved frozen under dry ice until immediately before oral administration. SFB colonizations were performed by oral gavage with 300-400 μl of suspension obtained by homogenizing the fecal pellets from SFB-mono donor mice in water. Control mice were gavaged with water or homogenates prepared from their own feces.

Cell Isolation and Flow Cytometry

Lamina propria lymphocyte (LPL) isolation and intracellular cytokine staining were performed as described before (Ivanov et al., 2008). Naive CD4⁺ T cells were purified from spleens using a CD4⁺CD62L⁺ T cell isolation kit II (Miltenyi Biotec; purity 95%). Anti-mouse RORγ monoclonal antibody conjugated to PE was purchased from eBioscience.

In Vitro T-Cell Differentiation

Naive CD4⁺ T cells were cultured in 24-well plates at 2×10⁵ cells/well for 4 days with MACS-purified LP CD11c⁺ cells (1×10⁵/well) and 1 μg/ml anti-CD3 antibody (BD Biosciences) in the presence or absence of recombinant human Apo-SAA (Peprotech Inc. Princeton Business Park, 5 Crescent Ave. Rocky Hill, N.J. 08553). The cultured cells were harvested and restimulated with PMA and ionomycin for 3 h before analysis.

Electron Microscopy

Dissected 0.5 cm piece of the terminal ileum was cut open, fixed in fixative containing 2.5% glutaraldehyde, and 2% paraformaldehyde in 0.1M sodium cacodylate buffer (pH 7.2) for 2 hours and post-fixed with 1% osmium tetroxide for 1.5 hours at room temperature, then processed in a standard manner and embedded in EMbed 812 (Electron Microscopy Sciences, Hatfield, Pa.) for transmission electron microscopy (TEM) or critical point dried for scanning electron microscopy (SEM). For TEM, semi-thin sections were cut at 1000 nm and stained with 1% Toluidine Blue to evaluate the quality of preservation. Ultrathin sections (60 nm) were cut, mounted on copper grids and stained with uranyl acetate and lead citrate by standard methods. Stained grids were examined using a Philips CM-12 electron microscope (FEI; Eindhoven, The Netherlands) and photographed with a Gatan (4k x2.7k) digital camera (Gatan, Inc., Pleasanton, Calif.). For SEM, critical point dried samples were quickly transferred to the SEM chamber to avoid contamination. No conductive coating was applied.

Electron Microscopy

Electron microscopy was performed on 0.5-1 cm pieces from terminal ileum (immediately proximal to the ileal-cecal junction). Tissue processing for EM is described herein above. The analysis was performed with a Zeiss Supra 55 FESEM.

C. rodentium Infection

IQI mice were inoculated with 200 μl of a bacterial suspension (1-2×10⁹ CFU) by way of oral gavage. For the colony formation assays, proximal colons and MLNs were harvested and homogenized, and serially diluted homogenates were plated on MacConkey agar (Difco). For histological analysis, distal colons were fixed with 4% paraformaldehyde and analyzed after haematoxylin and eosin staining. For assessment of crypt depth, only crypts visible along the entire length of the distal colon were analyzed (20˜30 crypts/mouse).

Results

### Comparative Analysis of the Intestinal Microbiota of Jackson and Taconic B6 Mice

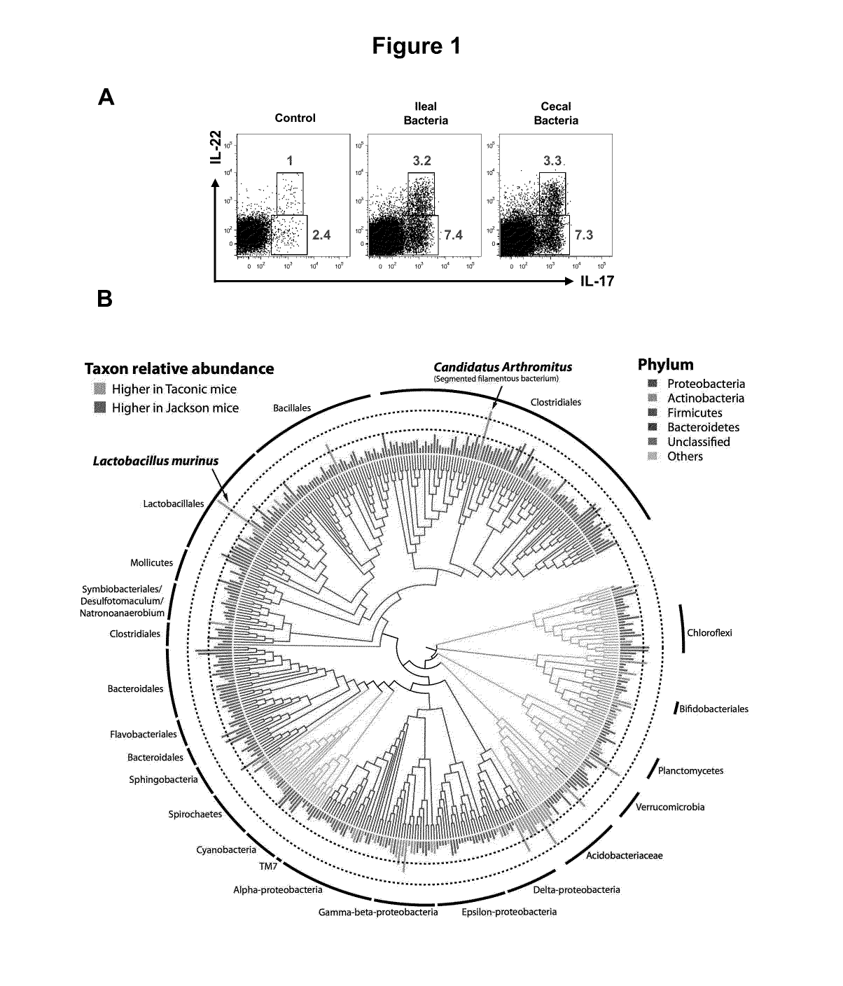

To identify the bacterial species that induce Th17 cells in the small intestine, we compared the bacterial content in B6 mice purchased from Taconic Farms and Jackson Laboratory. We previously showed that transfer of cecal contents could induce Th17 cells in recipient mice (Ivanov et al., 2008). Because we find more Th17 cells in the small intestine than in the large intestine (LI), we surmised that the Th17 cell-inducing bacterial species are present also in the small intestinal microbiota. Indeed, colonization of GF and Jackson B6 mice with the contents of the small intestines of Taconic B6 mice induced numbers of Th17 cells similar to those in specific-pathogen-free (SPF) Taconic B6 mice (FIG. 1A). We therefore chose to investigate the bacterial composition of the small intestine in detail. To provide an in-depth profile of the bacterial communities present in the small intestine of Taconic and Jackson mice, we analyzed these samples using the 16S rRNA PhyloChip (Brodie et al., 2006), a high-density microarray with over 300,000 probes targeting the sequence polymorphisms in the 16S rRNA gene, permitting detection of approximately 8,500 bacterial taxa. Overall 1,164 taxa were detected across all samples, 509±32 taxa were detected in Jackson B6 mice and 828±82 taxa in Taconic B6 mice. Our previous analysis by FISH had demonstrated a correlation between a probe for the Cytophaga-Flavobacter-Bacteroides (CFB) phylum and the presence of Th17 cells in Taconic mice (Ivanov et al., 2008). However, closer analysis of recently published 16S rRNA sequences revealed that this probe also matches perfectly to a number of non-CFB taxa, including bacteria in the Firmicutes, Actinobacteria, and Verrucomicrobia phyla. The PhyloChip analysis demonstrated that, indeed, the overall representation of the major bacterial phyla, including CFB, was not statistically different between the two mouse strains.

The depth of coverage and phylogenetic breadth of the PhyloChip allowed us to assay the microbial community at multiple phylogenetic levels, while its sensitivity permitted detection of less abundant organisms even in dominated communities (DeSantis et al., 2007). Comparative analysis of 766 bacterial taxa detected in at least 3 out of 4 replicates from either strain of mice demonstrated that the relative abundance of 479 taxa was significantly different (p<0.05) between the two mouse strains, with 372 taxa having greater abundance in Jackson mice and 107 taxa overrepresented in the Taconic group. However, of the 479 significantly different taxa, most differences were subtle, with only 52 being above 5-fold (17 greater in Taconic and 35 greater in Jackson) and only two taxa were >25-fold more abundant. These were identified as members of the Lactobacillaceae and Clostridiaceae families—Lactobacillus murinus ASF361 and a segmented filamentous species of the candidate genus Arthromitus (FIG. 1B). Both were of significantly greater (p<0.001) relative abundance in Taconic mice (˜94-fold for Lactobacillus murinus and ˜40-fold for Candidatus Arthromitus); however, since both were below the PhyloChip threshold of detection in the Jackson mice, these fold changes should be considered a minimum. For both taxa, overrepresentation of close phylogenetic relatives was not observed, suggesting a species-specific increase in relative abundance (FIG. 1B).

Lactobacillus murinus ASF361 is a component of the ASF (Dewhirst et al., 1999). ASF is used by Taconic Farms as a basal inoculum introduced into all Taconic re-derived strains, but is not intentionally introduced into Jackson Laboratory animals. Because of these differences, we previously tested if L. murinus ASF361, in the context of ASF, induces Th17 cell differentiation. Colonization of germ-free mice with ASF, including L. murinus ASF361, did not induce any Th17 cells in the SI LP (Ivanov et al., 2008). We therefore concluded that L. murinus ASF 361 is not involved in the induction of Th17 cell differentiation.

Presence of SFB Correlates with the Presence of Th17 Cells

We next examined the representation of Candidatus Arthromitus in Th17 cell-sufficient and Th17 cell-deficient mice. Arthromitus is an unofficial candidate genus name for the group of so-called segmented filamentous bacteria (Snel et al., 1995). SFB are yet to be cultured, commensal, gram-positive, anaerobic, spore-forming bacteria that are resident in the terminal ileum under steady state conditions (Davis and Savage, 1974). SFB have a characteristic long filamentous morphology, are comprised of multiple segments with well-defined septa, and often span the length of several villi. They colonize the gastrointestinal tract of mice at weaning time and adhere tightly to epithelial cells (Koopman et al., 1987). SFB are present in a many vertebrate species, including rodents (Davis and Savage, 1974), fish, chicken, dogs, and primates (Klaasen et al., 1993a; Ley et al., 2008). SFB are known to actively interact with the immune system (Klaasen et al., 1993b). Colonization of germ-free animals with SFB leads to stimulation of secretory IgA (SIgA) production and recruitment of intraepithelial lymphocytes (IELs) to the gut (Talham et al., 1999; Umesaki et al., 1999). Mice lacking the activation-induced cytidine deaminase (AID) required for antibody diversification had outgrowth of SFB in their small intestine (Suzuki et al., 2004).

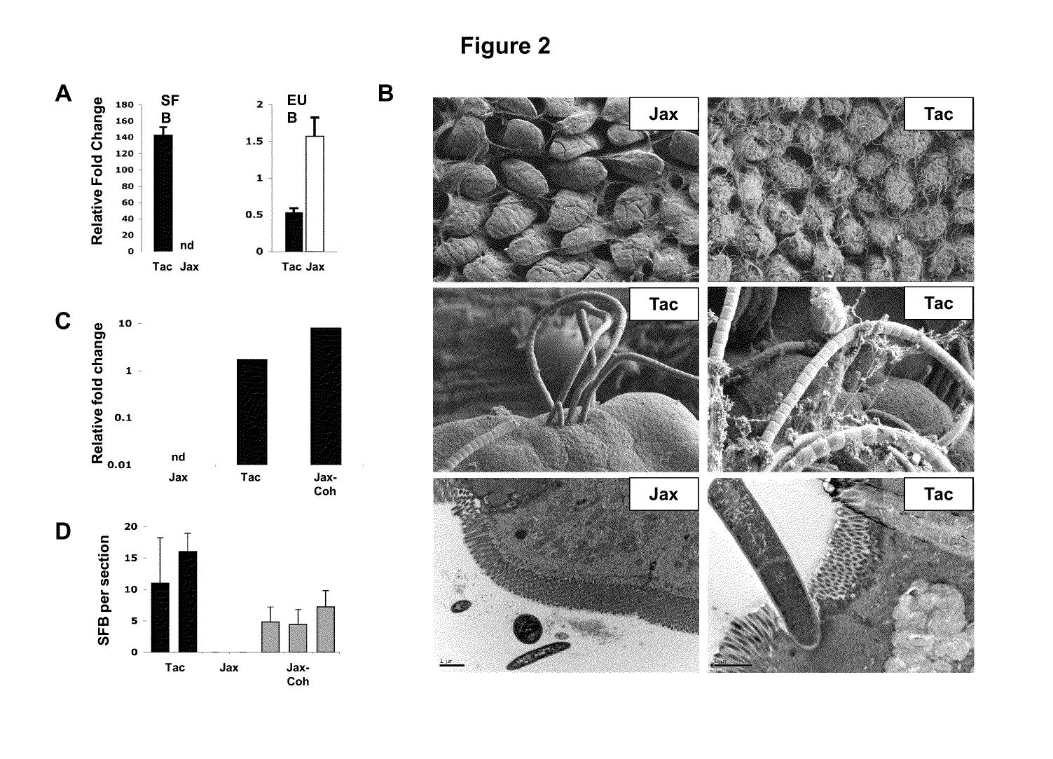

We validated the abundance of SFB in the gut of Taconic and Jackson B6 mice by quantitative real-time PCR (qPCR) for 16S rDNA sequences. SFB were present in fecal material from cecum as well as small and large intestine of Taconic B6 mice, but could not be detected in Jackson B6 mice (FIG. 2A). Scanning electron microscopy revealed a thick network of SFB present in the terminal ileum of 6-8 week old Taconic B6 mice (FIG. 2B). In contrast we could not detect any bacteria with SFB morphology in age- and sex-matched Jackson B6 mice, even after equilibration of housing conditions and diet (FIG. 2B). Despite similar numbers of total bacteria in the feces of both strains, only non-SFB bacteria were evident in the terminal ileum of mice from Jackson Laboratory. Transmission electron microscopy confirmed typical SFB morphology with well-defined segments in tight contact with the epithelial cells of ileum from Taconic but not Jackson B6 mice (FIG. 2B). To confirm that SFB can be horizontally transferred, we co-housed female mice obtained from the two sources and observed Th17 cells in the lamina propria of Jackson B6 mice within 10 days ((Ivanov et al., 2008) and data not shown). qPCR analysis of fecal material and microscopy of terminal ileum confirmed the appearance of SFB in the co-housed Jackson B6 mice (FIGS. 2C and 2D).

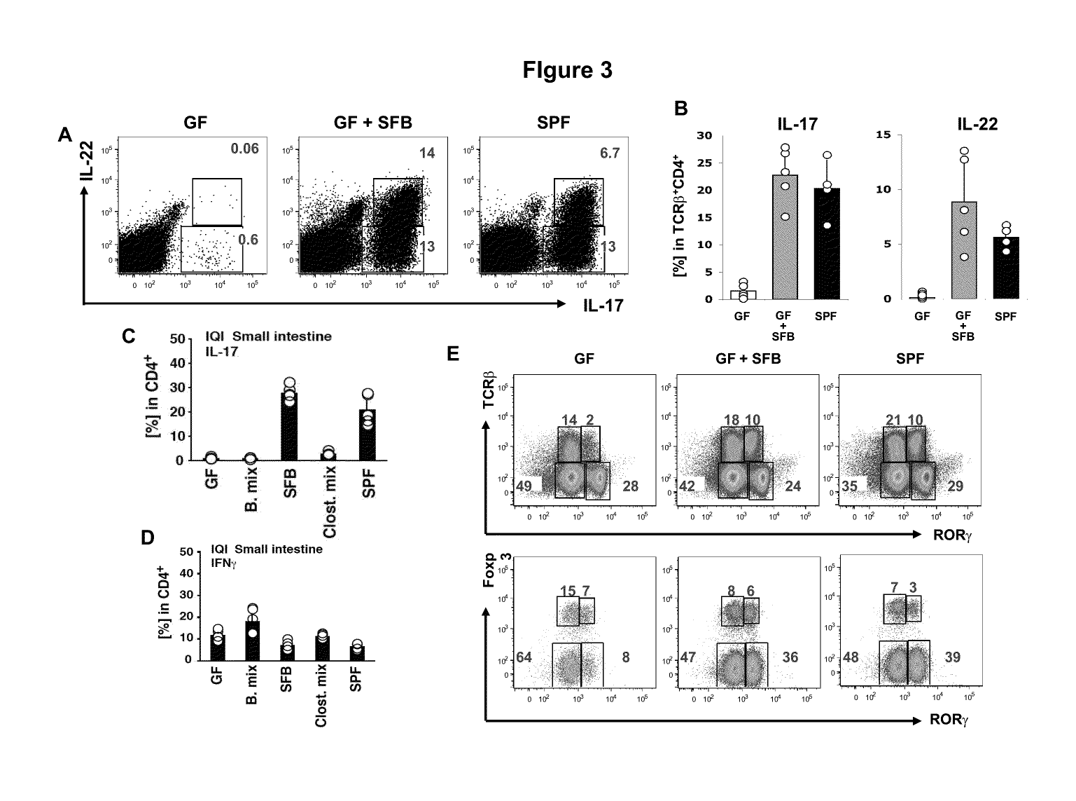

SFB specifically induce Th17 cells in the intestinal lamina propria To test if SFB are sufficient to induce Th17 cells, we colonized germ-free (GF) Swiss-Webster mice with fecal material obtained from mice mono-colonized with SFB (SFB-mono mice) (Umesaki et al., 1995) and examined lamina propria CD4⁺ T cells for Th17 cell differentiation 10 days later. Non-colonized control GF mice housed under separate but similar conditions had no Th17 cells (FIG. 3A). In contrast, SFB colonization induced robust accumulation of Th17 cells in both the SI and LI LP (FIG. 3A). SFB induced production of both IL-22 and IL-17 in CD4⁺ T cells (FIGS. 3A and 3B). The effect of SFB on Th17 cell differentiation was similar in Swiss-Webster and IQI GF mice housed at different institutions (FIGS. 3B and 3C). Moreover, the effect of SFB on inducing IL-17 production in LP T cells is bacterial species specific, because colonization with Bacteroides species as well as with a defined mix of Clostridium species, which are closely related to SFB, did not induce Th17 cells in GF mice (FIG. 3C). Finally, SFB had no effect on IFN-γ production, indicating that they specifically influence Th17 and not Th1 cell differentiation (FIG. 3D). Colonization of GF mice with SFB restored RORγt′ T cells to the levels observed in mice kept under SPF conditions (FIG. 3E). By contrast, the number of RORγt⁺ non-T cells, which include lymphoid tissue inducer-like cells and NK-like cells, was similar in GF mice, SFB-mono mice, and mice kept in SPF conditions (FIG. 3E), and there was no significant difference in IL-17 and IL-22 production by these cells. Notably, SFB colonization and induction of Th17 cells did not reverse the elevated proportion of Foxp3⁺ cells among the CD4⁺ T cells in the SI LP and the peritoneal cavity of GF mice.

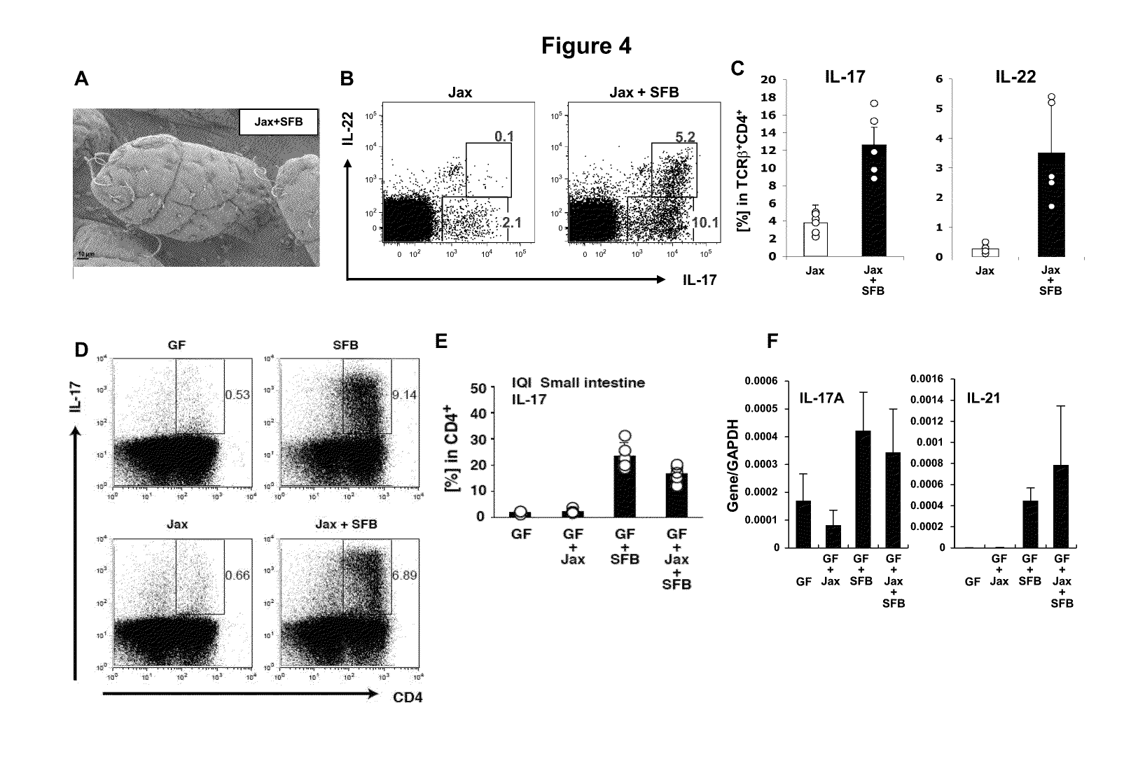

To determine whether SFB can also induce Th17 cell differentiation in conventionally raised mice, we introduced fecal material from SFB-mono mice by oral gavage into 6 week-old Jackson B6 mice and analyzed colonization and cytokine production in the SI LP. By 10 days, SFB were detected by scanning electron microscopy in the terminal ileum (FIG. 4A) and by qPCR in the feces (data not shown), and robust Th17 cell differentiation was observed in the SI LP (FIGS. 4B and 4C). In contrast, control untreated Jackson B6 mice or Jackson B6 mice gavaged with bacterial suspensions from their littermates did not show an increase in Th17 cells (FIGS. 4B and 4C). Similarly, introduction of Jackson microbiota into GF animals did not induce Th17 cells, unless the microbiota were supplemented with SFB (FIGS. 4D and 4E). Th17 cell induction by SFB was also demonstrated by the expression of a number of Th17 cell effector cytokine mRNAs, including those for IL-17 and IL-21 (FIG. 4F). We therefore conclude that SFB are members of the commensal microbiota that specifically induce the accumulation of Th17 cells in the SI LP.

SFB Induce an Immune Response Program in the Gut

To identify specific effects of SFB, we compared the gene expression profiles in the terminal ileum of Swiss-Webster GF mice before and after colonization with SFB and in Jackson B6 mice before and after co-housing with Taconic B6 animals. Colonization of GF mice with SFB induced at least a two-fold change in expression of 253 genes while co-housing of Jackson B6 mice with Taconic B6 mice induced a similar change in 470 genes (FIG. 5A). More importantly, there was a high degree of overlap between the two groups, with expression of 131 genes affected by both treatments. We could therefore distinguish three groups of genetic profiles. Group 1 includes genes whose expression was affected only in Jackson mice by co-housing, but was not statistically different after SFB colonization. This group most likely includes genes whose expression is influenced by microbiota other than SFB that differs between the mice from the different vendors, as well as strain-specific changes. Group 2 consists of genes whose expression only changed in GF mice upon colonization with SFB, but not in Jackson B6 mice following co-housing. A subset of these genes is expected to reflect changes induced in GF animals upon general intestinal colonization with bacteria. Group 3 includes the genes with expression differences following both SFB colonization and co-housing with Taconic mice (FIG. 5A) and thus contains genes specifically induced by SFB and associated with Th17 cell induction.

SFB exerted an inductive effect in the host, which was demonstrated by the finding that most (>70%) of the genes in Group 3 were up-regulated after SFB colonization (FIG. 5B). By comparison, most genes in Group 1 (>70%) were down-regulated, which suggests that the rest of the Taconic microbiota has a suppressive effect that may possibly restrain the inductive effect of SFB (FIG. 5B). Group 2, on the other hand, consisted of roughly equal numbers of up-regulated and down-regulated genes.

To evaluate changes specifically associated with Th17 cell-inducing SFB, we next concentrated on the genes in Group 3. A list of the top up-regulated genes is presented in FIG. 5F. A GO Biological Pathway analysis of up-regulated genes in Group 3 showed that immune system pathways were among the programs most significantly induced by SFB (FIG. 5C) and raised the possibility that at least some of the observed gene expression changes were mediated by Th17 cells or their effector cytokines Because IL-17 and IL-22 have been associated with induction of anti-microbial peptides (AMP) (Curtis and Way, 2009; Kolls et al., 2008; Zheng et al., 2008), we compared the induction of AMP-related genes in our arrays. Multiple AMP genes were induced specifically by colonization with SFB, consistent with an up-regulated Th17 cell response (FIG. 5D). Upregulation of Th17 cell-associated genes (Il17, Il21, Ccr6, Nos2) and AMPs (Reg3g) following SFB colonization was confirmed by quantitative RT-PCR (FIGS. 4F and 5E).

Serum Amyloid A is Induced by SFB Colonization and Influences Th17 Cell Differentiation

The top up-regulated transcript upon SFB colonization of GF mice encoded an isoform of SAA—Saa1, a member of the family of acute-phase response proteins induced during infection, tissue damage, or inflammatory disease (Uhlar and Whitehead, 1999). This transcript was also up-regulated upon co-housing of Jackson B6 mice with Taconic B6 animals (FIG. 5F). Transcripts for the other SAA isoforms, Saa2 and Saa3, were also among the most highly up-regulated genes upon colonization with SFB or co-housing (FIG. 5F).

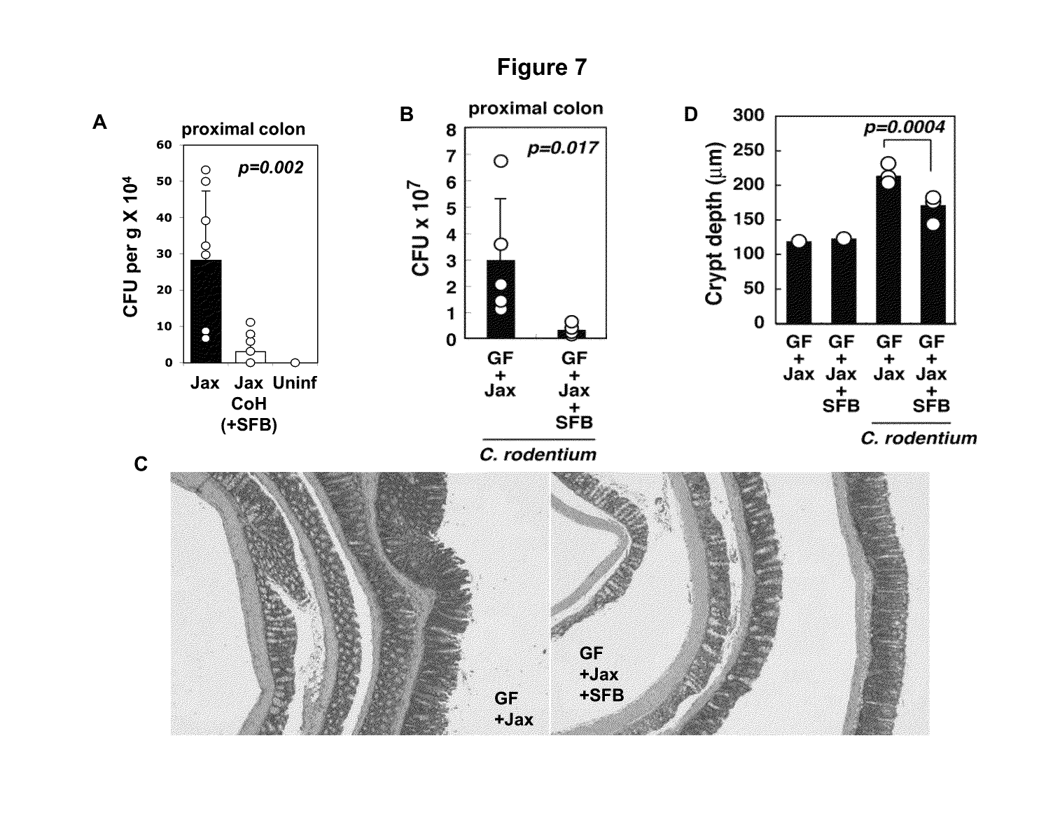

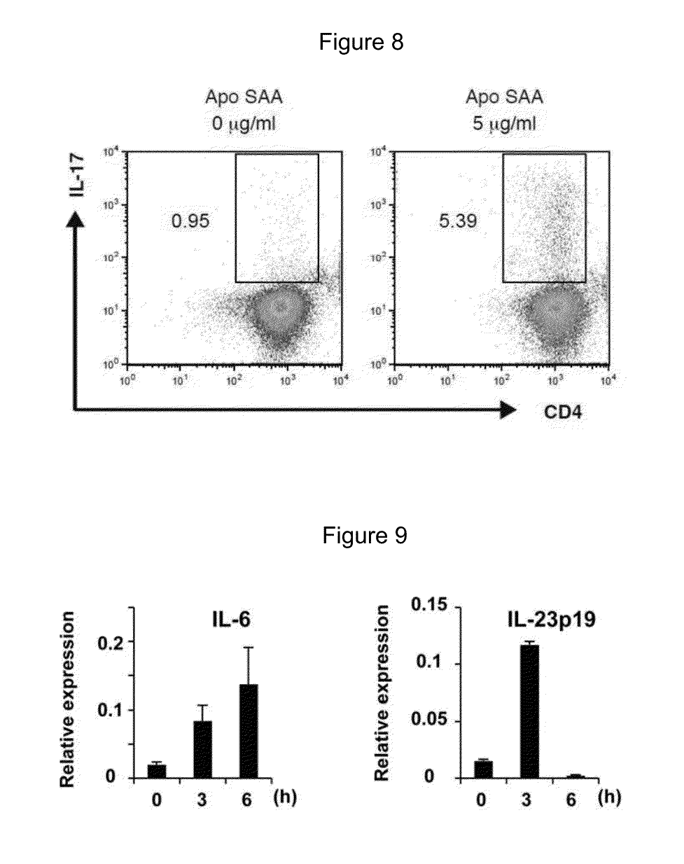

Real-time PCR confirmed that all three SAA isoforms were induced in the terminal ileum of GF mice upon colonization with SFB or SFB+Jackson microbiota, but not by Jackson microbiota alone (FIG. 6A). Recent studies have demonstrated that SAA may act as a cytokine that induces IL-8, TNFα, and IL-1β in neutrophils and IL-23 in monocytes (Furlaneto and Campa, 2000; He et al., 2006). We therefore investigated the effect of SAA on Th17 cell differentiation in vitro. Addition of recombinant SAA to co-cultures of naive CD4⁺ T cells and LP DCs induced a Th17 cell differentiation program in a concentration-dependent manner, including Th17 cell effector cytokines and RORγt (FIG. 6B). In addition, SAA induced production of IL-17 in CD4⁺ splenic OT-II T cells co-cultured with LP DCs in vitro (FIG. 8). Addition of SAA to cultures containing only T cells, without DCs, did not induce Th17 cell cytokines (FIG. 6B) and SAA induced production of IL-6 and IL-23 by LP DCs in vitro (FIG. 9). We conclude that SFB colonization results in the production of SAA, which in turn acts on gut DCs to stimulate a Th17 cell-inducing environment.

SFB Colonization Reduces Growth of an Intestinal Pathogen

We next examined the effect of Th17 cell-inducing microbiota and SFB on oral infection with Citrobacter rodentium, an intestinal pathogen whose clearance by the host requires an immune response dependent on IL-23, IL-22, and RegIIIγ (Mangan et al., 2006; Torchinsky et al., 2009; Zheng et al., 2008). Jackson B6 mice that had been co-housed with Taconic B6 mice and hence were colonized with SFB were significantly more resistant to growth of C. rodentium compared to non-co-housed mice, as demonstrated by recovery of infectious units from the wall of the colon (FIG. 7A).

To assess specifically the ability of SFB to provide protection, we colonized GF IQI mice with Jackson microbiota with or without SFB for 14 days. The mice were then infected orally with C. rodentium and pathogen colonization and disease were assessed at day 8 post-infection. Although some infection and disease were observed in both experimental groups, the presence of SFB in the gut prevented infiltration of the pathogen into the colonic wall (FIG. 7B). In addition, SFB colonization ameliorated colonic inflammation as demonstrated by reduced epithelial hyperplasia and colon shortening in its presence (FIG. 7C, 7D). We thus conclude that the presence of SFB as a component of the commensal microbiota increases mucosal protection to infection with C. rodentium.

### DISCUSSION

Commensal Intestinal Bacteria and Regulation of Th17 Cell Differentiation Commensal intestinal bacteria influence multiple metabolic and physiological functions of the host (Backhed et al., 2005; Turnbaugh et al., 2006), but they also have profound effects on the host immune system (Cash et al., 2006; Macpherson and Harris, 2004). For example, most rodent colitis models are dependent on the presence of microbiota (Elson et al., 2005; Sartor, 2008), whose products can also influence systemic immune responses (Mazmanian et al., 2005; Turnbaugh et al., 2006). The effects of intestinal bacteria on the immune system are considered to be the result of stimulation of innate immune “pattern recognition receptors”, but we are limited in our understanding of how individual bacteria influence the type and location of immune responses in gnotobiotic models or in the presence of other commensal microorganisms (Macpherson and Harris, 2004; Umesaki et al., 1999) (Kim et al., 2005). A notable exception was the recent demonstration that a polysaccharide product from the commensal bacterium Bacteroides fragilis can specifically induce systemic Th1 and mucosal regulatory T cell responses and protect mice from pathogen-induced colitis (Mazmanian et al., 2005; Mazmanian et al., 2008).

We recently discovered that the homeostasis of effector helper T cell populations in the gut is dependent on the composition of intestinal bacteria (Ivanov et al., 2008). Th17 cell induction was not controlled simply by the presence of high numbers of diverse bacterial species that activate major bacterial pattern-recognition pathways. Thus, colonization of mice with several defined bacterial species as well as with diverse microbiota from Jackson Laboratory B6 mice did not induce Th17 cell differentiation, in sharp contrast to the induction observed with bacteria from Th17 cell-sufficient Taconic Farms B6 mice (Ivanov et al., 2008). The identity of microorganisms that induce Th17 cells and the signaling mechanisms involved had remained important unresolved issues.

In this study, we have identified SFB as the first commensal bacterium that can induce accumulation in the gut of CD4⁺ T cells with a defined effector function. Colonization with a number of other species, including members of the SFB-related Clostridiaceae family, failed to induce Th17 cells. Our results are most consistent with a mechanism that requires unique features of a specific commensal species to trigger Th17 cell differentiation and/or accumulation in the lamina propria. Pathways activated by common bacterial patterns and shared by large classes of bacteria appear to be dispensable or redundant, as both MyD88/TRIF double deficient animals and RIP-2 mutant mice still possessed mucosal Th17 cells (Atarashi et al., 2008; Ivanov et al., 2008). We previously reported that adenosine 5′-triphosphate (ATP) derived from commensal bacteria led to the differentiation of Th17 cells in the colonic LP (Atarashi et al., 2008). However, the ATP concentrations in the ileal and colonic luminal contents of SFB-mono mice were lower than those in mice gavaged with SPF feces. Thus, SFB-mediated Th17 cell differentiation is likely to occur through a mechanism independent of TLR-, NOD-, and ATP-signaling.

SFB associate closely with epithelial cells in the terminal ileum. This interaction was reflected in the host genes induced after SFB colonization. Multiple epithelial cell-specific genes, as well as inflammatory response host genes, were up-regulated by the bacteria. Among these were the three inducible or “acute-phase” isoforms of SAA (A-SAA). SAA is highly induced during both acute and chronic inflammation. A-SAA expression is induced in hepatocytes in the liver and in macrophages and other cells in extrahepatic sites, including the intestine, by bacterial products and inflammatory cytokines, such as IL-6 and IL-1β (Uhlar and Whitehead, 1999). In addition to its role in the acute-phase response, SAA can induce IL-23 production by monocytes at concentrations that are orders of magnitude lower than the peak plasma concentration during an acute-phase response (He et al., 2006). In accordance with this, SAA induced transient production of IL-23 by LP DCs in vitro. We further demonstrated that SAA can act on LP DCs in vitro to induce Th17 cell differentiation, suggesting that an acute phase inflammation-like response, including induction of A-SAAs, is responsible for the SFB-mediated accumulation of Th17 cells in the intestine. Although the signaling pathways induced by A-SAA are currently unknown, it most likely acts on DCs and contributes to the establishment of a Th17 cell-inducing cytokine environment.

SFB and Th17 Cell-Mediated Protection from Pathogenic Microorganisms

The identification of SFB as Th17 cell inducers in the intestine may have important implications for a better understanding of how components of the commensal microbiota contribute to host protection from microbial pathogens. It is well known that treatment with broad-spectrum antibiotics can result in outgrowth of intestinal pathogens, such as vancomycin-resistant Enterococcus (VRE) or Clostridium difficile, resulting in severe colitis. SFB colonization has been suggested to reduce replication in rabbits of enteropathogenic Escherichia coli (EPEC) and in rats of Salmonella enteritidis (Garland et al., 1982; Heczko et al., 2000). In mice, Th17 cell effector cytokines, such as IL-17 and IL-22, as well as IL-23, which is required for Th17 cell function, have been proposed to play protective roles in infections with Salmonella and Citrobacter rodentium (Curtis and Way, 2009). We found that colonization with SFB reduced the capacity of orally inoculated C. rodentium to grow and/or invade colonic tissue. Although we cannot at this point formally demonstrate that this protection is a direct result of Th17 cell induction, our data, taken together with results of recent studies (Kolls et al., 2008; Zheng et al., 2008), strongly suggest that SFB-induced Th17 cytokines, particularly IL-22, limit the growth of C. rodentium, at least in part through production of AMP's such as RegIIIγ. While IL-22, IL-23, and RegIIIγ are required for host survival after C. rodentium infection (Mangan et al., 2006; Zheng et al., 2008), mice lacking SFB and Th17 cells survive despite increased bacterial growth. This may be because intestinal γδ T cells, CD4⁺CD3⁻ lymphoid tissue inducer (LTi)-like cells, and NK22 cells that also produce Th17 cytokines are present even in the absence of SFB and other microbiota. Contribution of these cells to SFB-independent anti-microbial defense may hence protect the host from lethal outgrowth of the pathogenic bacteria.

Our results are also consistent with the report that a vancomycin-sensitive component of the commensal microbiota induces RegIIIγ in the mouse small intestine, thus reducing colonization by VRE and enhancing killing of the pathogen (Brandl et al., 2008). Future studies will be required to determine if vancomycin-sensitive SFB enhance mucosal protection from pathogenic VRE and other bacteria through the up-regulation of Th17 cells and anti-microbial peptides. Such studies will further test the hypothesis that specific commensal microbiota, by regulating the host immune system rather than by direct microbial competition, enhance protection from potentially harmful microbes.

Do SFB Influence Th17 Cell-Mediated Inflammatory Disease?

Th17 cells are recognized to have significant roles in multiple mouse models of autoimmune disease, and there is accumulating evidence that they likewise contribute to human autoimmune disease pathogenesis (Hue et al., 2006; Langrish et al., 2005; Murphy et al., 2003; Yen et al., 2006). Mice with almost complete loss of Th17 cells due to the absence of RORγt are resistant to experimental autoimmune encephalomyelitis and colitis (Ivanov et al., 2006; Leppkes et al., 2009). In humans, polymorphisms in the gene encoding the IL-23 receptor are associated with both increased resistance and susceptibility to Crohn's disease, and inhibition of the Th17 cell differentiation pathway has been reported to be an effective therapy for psoriasis (Duerr et al., 2006; Krueger et al., 2007).

Although Th17 cells are involved in multiple organ-specific inflammatory diseases, they are not normally present in such organs, and they are relatively scarce in secondary lymphoid tissues. However, Th17 cells are abundant in the intestinal lamina propria, and, as described in this study, their differentiation within and/or migration to this lymphoid-rich site is dependent on commensal microbes with specialized properties. There is evidence that the course of certain autoimmune diseases in humans and in animal models can be altered by treatment with antibiotics and probiotics and by restricting the complexity of the microbiota (O'Dell et al., 2006; Sartor, 2008). Indeed, in rodents, differential arthritogenic potential of different commensal microbiota components and dependence of spontaneous arthritis models on “cleanliness” of housing conditions have been reported (Severijnen et al., 1989; Simelyte et al., 2003). Moreover, K/B×N mice that have a genetic predisposition to spontaneous arthritis (Monach et al., 2008) fail to develop disease when kept in GF conditions, but do progress to arthritis when colonized with SFB (Wu et al., Immunity 32:815; 2010). Our results thus raise the possibility that manipulation of the number of SFB that colonize the terminal ileum may alter the course of Th17 cell-associated autoimmune diseases.

If Th17 cells involved in organ-specific autoimmunity originate in the gut, then the question arises as to what is the antigenic specificity of such cells. It is not yet known if Th17 cells in the lamina propria are specific for intestinal microbiota. If they are mostly reactive with microbial products, then it may be surprising that similar numbers of Th17 cells are observed in mice monocolonized with SFB and in mice with a broad distribution of microbiota. Th17 cells specific for bacterial products may constitute a sufficiently broad repertoire to provide subsets that are cross-reactive with self-antigen. Alternatively, intestinal Th17 cells may be broadly specific for self-antigen, rather than bacterial products, but may normally be kept in check by mechanisms of peripheral tolerance. Signals from bacteria such as SFB may provide an adjuvant effect that polarizes such self-reactive T helper cells towards the Th17 lineage without tissue damage under the immune suppressive environment in the gut. Further studies on the repertoire and antigen specificity of Th17 cells and on the role of SFB in autoimmune disease models will be necessary to resolve these issues.

SFB represent the first example of a specific component of the commensal microbiota that induces a particular helper T cell population in the lamina propria. The elucidation of additional commensal bacteria involved in this or other immune pathways and of the mechanisms employed will undoubtedly lead to further understanding of the complex host-commensal interactions that shape our immunity and will allow for tailored therapeutic manipulation of these processes.

### REFERENCES

Abbas, A. K., Murphy, K. M., and Sher, A. (1996). Functional diversity of helper T lymphocytes. Nature 383, 787-793.
Atarashi, K., Nishimura, J., Shima, T., Umesaki, Y., Yamamoto, M., Onoue, M., Yagita, H., Ishii, N., Evans, R., Honda, K., et al. (2008). ATP drives lamina propria T(H)17 cell differentiation. Nature 455, 808-812.
Aujla, S. J., Dubin, P. J., and Kolls, J. K. (2007). Th17 cells and mucosal host defense. Semin Immunol 19, 377-382.
Backhed, F., Ley, R. E., Sonnenburg, J. L., Peterson, D. A., and Gordon, J. I. (2005). Host-bacterial mutualism in the human intestine. Science 307, 1915-1920.
Barman, M., Unold, D., Shifley, K., Amir, E., Hung, K., Bos, N., and Salzman, N. (2008). Enteric salmonellosis disrupts the microbial ecology of the murine gastrointestinal tract. Infect Immun 76, 907-915.
Bettelli, E., Oukka, M., and Kuchroo, V. K. (2007). T(H)-17 cells in the circle of immunity and autoimmunity. Nat Immunol 8, 345-350.
Brandl, K., Plitas, G., Mihu, C. N., Ubeda, C., Jia, T., Fleisher, M., Schnabl, B., DeMatteo, R. P., and Pamer, E. G. (2008). Vancomycin-resistant enterococci exploit antibiotic-induced innate immune deficits. Nature 455, 804-807.
Brodie, E. L., Desantis, T. Z., Joyner, D. C., Baek, S. M., Larsen, J. T., Andersen, G. L., Hazen, T. C.,
Richardson, P. M., Herman, D. J., Tokunaga, T. K., et al. (2006). Application of a high-density oligonucleotide microarray approach to study bacterial population dynamics during uranium reduction and reoxidation. Appl Environ Microbiol 72, 6288-6298.
Brodie, E. L., T. Z. Desantis, et al. (2007). “Urban aerosols harbor diverse and dynamic bacterial populations.” Proc Natl Acad Sci USA 104(1): 299-304.
Cash, H. L., Whitham, C. V., Behrendt, C. L., and Hooper, L. V. (2006). Symbiotic bacteria direct expression of an intestinal bactericidal lectin. Science 313, 1126-1130.
Curtis, M. M., and Way, S. S. (2009). Interleukin-17 in host defence against bacterial, mycobacterial and fungal pathogens. Immunology 126, 177-185.
Davis, C. P., and Savage, D. C. (1974). Habitat, succession, attachment, and morphology of segmented, filamentous microbes indigenous to the murine gastrointestinal tract. Infect Immun 10, 948-956.
Dennis, G., Jr., Sherman, B. T., Hosack, D. A., Yang, J., Gao, W., Lane, H. C., and Lempicki, R. A. (2003). DAVID: Database for Annotation, Visualization, and Integrated Discovery. Genome biology 4, P3.
DeSantis, T. Z., Brodie, E. L., Moberg, J. P., Zubieta, I. X., Piceno, Y. M., and Andersen, G. L. (2007). High-density universal 16S rRNA microarray analysis reveals broader diversity than typical clone library when sampling the environment. Microb Ecol 53, 371-383.
DeSantis, T. Z., P. Hugenholtz, et al. (2006). “Greengenes, a chimera-checked 16S rRNA gene database and workbench compatible with ARB.” Applied and Environmental Microbiology 72(7): 5069-5072.
Dewhirst, F. E., Chien, C. C., Paster, B. J., Ericson, R. L., Orcutt, R. P., Schauer, D. B., and Fox, J. G. (1999). Phylogeny of the defined murine microbiota: altered Schaedler flora. Appl Environ Microbiol 65, 3287-3292.
Duerr, R. H., Taylor, K. D., Brant, S. R., Rioux, J. D., Silverberg, M. S., Daly, M. J., Steinhart, A. H., Abraham, C., Regueiro, M., Griffiths, A., et al. (2006). A genome-wide association study identifies IL23R as an inflammatory bowel disease gene. Science 314, 1461-1463.
Elson, C. O., Cong, Y., McCracken, V. J., Dimmitt, R. A., Lorenz, R. G., and Weaver, C. T. (2005). Experimental models of inflammatory bowel disease reveal innate, adaptive, and regulatory mechanisms of host dialogue with the microbiota. Immunol Rev 206, 260-276.
Fontenot, J. D., Gavin, M. A., and Rudensky, A. Y. (2003). Foxp3 programs the development and function of CD4+CD25+ regulatory T cells. Nat Immunol 4, 330-336.
Furlaneto, C. J., and Campa, A. (2000). A novel function of serum amyloid A: a potent stimulus for the release of tumor necrosis factor-alpha, interleukin-lbeta, and interleukin-8 by human blood neutrophil. Biochemical and biophysical research communications 268, 405-408.
Garland, C. D., Lee, A., and Dickson, M. R. (1982). Segmented Filamentous Bacteria in the Rodent Small Intestine: Their Colonization of Growing Animals and Possible Role in Host Resistance to Salmonella. Microb Ecol, 181-190.
Gavin, M., and Rudensky, A. (2003). Control of immune homeostasis by naturally arising regulatory CD4+ T cells. Curr Opin Immunol 15, 690-696.
Glimcher, L. H., and Murphy, K. M. (2000). Lineage commitment in the immune system: the T helper lymphocyte grows up. Genes Dev 14, 1693-1711.
Hall, J. A., Bouladoux, N., Sun, C. M., Wohlfert, E. A., Blank, R. B., Zhu, Q., Grigg, M. E., Berzofsky, J. A., and Belkaid, Y. (2008). Commensal DNA limits regulatory T cell conversion and is a natural adjuvant of intestinal immune responses. Immunity 29, 637-649.
He, R., Shepard, L. W., Chen, J., Pan, Z. K., and Ye, R. D. (2006). Serum amyloid A is an endogenous ligand that differentially induces IL-12 and IL-23. J Immunol 177, 4072-4079.
Heczko, U., Abe, A., and Finlay, B. B. (2000). Segmented filamentous bacteria prevent colonization of enteropathogenic Escherichia coli O103 in rabbits. The Journal of infectious diseases 181, 1027-1033.
Hori, S., Nomura, T., and Sakaguchi, S. (2003). Control of regulatory T cell development by the transcription factor Foxp3. Science 299, 1057-1061.
Huang da, W., Sherman, B. T., and Lempicki, R. A. (2009). Systematic and integrative analysis of large gene lists using DAVID bioinformatics resources. Nature protocols 4, 44-57.
Hue, S., Ahern, P., Buonocore, S., Kullberg, M. C., Cua, D. J., McKenzie, B. S., Powrie, F., and Maloy, K. J. (2006). Interleukin-23 drives innate and T cell-mediated intestinal inflammation. J Exp Med 203, 2473-2483.
Itoh, K., and Mitsuoka, T. (1985). Characterization of clostridia isolated from faeces of limited flora mice and their effect on caecal size when associated with germ-free mice. Laboratory animals 19, 111-118.
Ivanov, II, Frutos Rde, L., Manel, N., Yoshinaga, K., Rifkin, D. B., Sartor, R. B., Finlay, B. B., and Littman, D. R. (2008). Specific microbiota direct the differentiation of IL-17-producing T-helper cells in the mucosa of the small intestine. Cell Host Microbe 4, 337-349.
Ivanov, II, McKenzie, B. S., Zhou, L., Tadokoro, C. E., Lepelley, A., Lafaille, J. J., Cua, D. J., and Littman, D. R. (2006). The orphan nuclear receptor RORgammat directs the differentiation program of proinflammatory IL-17+ T helper cells. Cell 126, 1121-1133.
Khattri, R., Cox, T., Yasayko, S. A., and Ramsdell, F. (2003). An essential role for Scurfin in CD4+CD25+ T regulatory cells. Nat Immunol 4, 337-342.
Kim, S. C., Tonkonogy, S. L., Albright, C. A., Tsang, J., Balish, E. J., Braun, J., Huycke, M. M., and Sartor, R. B. (2005). Variable phenotypes of enterocolitis in interleukin 10-deficient mice monoassociated with two different commensal bacteria. Gastroenterology 128, 891-906.
Klaasen, H. L., Koopman, J. P., Van den Brink, M. E., Bakker, M. H., Poelma, F. G., and Beynen, A. C. (1993a). Intestinal, segmented, filamentous bacteria in a wide range of vertebrate species. Laboratory animals 27, 141-150.
Klaasen, H. L., Van der Heijden, P. J., Stok, W., Poelma, F. G., Koopman, J. P., Van den Brink, M. E., Bakker, M. H., Eling, W. M., and Beynen, A. C. (1993b). A pathogenic, intestinal, segmented, filamentous bacteria stimulate the mucosal immune system of mice. Infect Immun 61, 303-306.
Kolls, J. K., McCray, P. B., Jr., and Chan, Y. R. (2008). Cytokine-mediated regulation of antimicrobial proteins. Nat Rev Immunol 8, 829-835.
Koopman, J. P., Stadhouders, A. M., Kennis, H. M., and De Boer, H. (1987). The attachment of filamentous segmented micro-organisms to the distal ileum wall of the mouse: a scanning and transmission electron microscopy study. Laboratory animals 21, 48-52.
Krueger, G. G., Langley, R. G., Leonardi, C., Yeilding, N., Guzzo, C., Wang, Y., Dooley, L. T., and Lebwohl, M. (2007). A human interleukin-12/23 monoclonal antibody for the treatment of psoriasis. The New England journal of medicine 356, 580-592.
Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240.
Leppkes, M., Becker, C., Ivanov, II, Hirth, S., Wirtz, S., Neufert, C., Pouly, S., Murphy, A. J., Valenzuela, D. M., Yancopoulos, G. D., et al. (2009). RORgamma-expressing Th17 cells induce murine chronic intestinal inflammation via redundant effects of IL-17A and IL-17F. Gastroenterology 136, 257-267.
Ley, R. E., Hamady, M., Lozupone, C., Turnbaugh, P. J., Ramey, R. R., Bircher, J. S., Schlegel, M. L., Tucker, T. A., Schrenzel, M. D., Knight, R., et al. (2008). Evolution of Mammals and Their Gut Microbes. Science.
Macpherson, A. J., and Harris, N. L. (2004). Interactions between commensal intestinal bacteria and the immune system. Nat Rev Immunol 4, 478-485.
Mangan, P. R., Harrington, L. E., O'Quinn, D. B., Helms, W. S., Bullard, D. C., Elson, C. O., Hatton, R. D., Wahl, S. M., Schoeb, T. R., and Weaver, C. T. (2006). Transforming growth factor-beta induces development of the T(H)17 lineage. Nature 441, 231-234.
Masuda, N. and G. M. Church (2002). “Escherichia coli gene expression responsive to levels of the response regulator EvgA.” J Bacteriol 184(22): 6225-34.
Mazmanian, S. K., Liu, C. H., Tzianabos, A. O., and Kasper, D. L. (2005). An immunomodulatory molecule of symbiotic bacteria directs maturation of the host immune system. Cell 122, 107-118.
Mazmanian, S. K., Round, J. L., and Kasper, D. L. (2008). A microbial symbiosis factor prevents intestinal inflammatory disease. Nature 453, 620-625.
Miyamoto, Y., and Itoh, K. (2000). Bacteroides acidifaciens sp. nov., isolated from the caecum of mice. International journal of systematic and evolutionary microbiology 50 Pt 1, 145-148.
Monach, P. A., Mathis, D., and Benoist, C. (2008). The K/B×N arthritis model. Current protocols in immunology/edited by John E Coligan [et al Chapter 15, Unit 15 22.
Murphy, C. A., Langrish, C. L., Chen, Y., Blumenschein, W., McClanahan, T., Kastelein, R. A., Sedgwick, J. D., and Cua, D. J. (2003). Divergent pro- and antiinflammatory roles for IL-23 and IL-12 in joint autoimmune inflammation. J Exp Med 198, 1951-1957.
O'Dell, J. R., Elliott, J. R., Mallek, J. A., Mikuls, T. R., Weaver, C. A., Glickstein, S., Blakely, K. M., Hausch, R., and Leff, R. D. (2006). Treatment of early seropositive rheumatoid arthritis: doxycycline plus methotrexate versus methotrexate alone. Arthritis Rheum 54, 621-627.
Rakoff-Nahoum, S., and Medzhitov, R. (2006). Role of the innate immune system and host-commensal mutualism. Curr Top Microbiol Immunol 308, 1-18.
Sartor, R. B. (2008). Microbial influences in inflammatory bowel diseases. Gastroenterology 134, 577-594.
Seder, R. A., and Paul, W. E. (1994). Acquisition of lymphokine-producing phenotype by CD4+ T cells. Annu Rev Immunol 12, 635-673.
Severijnen, A. J., van Kleef, R., Hazenberg, M. P., and van de Merwe, J. P. (1989). Cell wall fragments from major residents of the human intestinal flora induce chronic arthritis in rats. The Journal of rheumatology 16, 1061-1068.
Simelyte, E., Rimpilainen, M., Zhang, X., and Toivanen, P. (2003). Role of peptidoglycan subtypes in the pathogenesis of bacterial cell wall arthritis. Annals of the rheumatic diseases 62, 976-982.
Snel, J., Heinen, P. P., Blok, H. J., Carman, R. J., Duncan, A. J., Allen, P. C., and Collins, M. D. (1995). Comparison of 16S rRNA sequences of segmented filamentous bacteria isolated from mice, rats, and chickens and proposal of “Candidatus Arthromitus”. International journal of systematic bacteriology 45, 780-782.
Suzuki, K., Meek, B., Doi, Y., Muramatsu, M., Chiba, T., Honjo, T., and Fagarasan, S. (2004). Aberrant expansion of segmented filamentous bacteria in IgA-deficient gut. Proc Natl Acad Sci USA 101, 1981-1986.
Talham, G. L., Jiang, H. Q., Bos, N. A., and Cebra, J. J. (1999). Segmented filamentous bacteria are potent stimuli of a physiologically normal state of the murine gut mucosal immune system. Infect Immun 67, 1992-2000.
Torchinsky, M. B., Garaude, J., Martin, A. P., and Blander, J. M. (2009) Innate immune recognition of infected apoptotic cells directs T(H)17 cell differentiation. Nature 458, 78-82.
Turnbaugh, P. J., Ley, R. E., Mahowald, M. A., Magrini, V., Mardis, E. R., and Gordon, J. I. (2006). An obesity-associated gut microbiome with increased capacity for energy harvest. Nature 444, 1027-1031.
Uhlar, C. M., and Whitehead, A. S. (1999). Serum amyloid A, the major vertebrate acute-phase reactant. European journal of biochemistry/FEBS 265, 501-523.
Umesaki, Y., Okada, Y., Matsumoto, S., Imaoka, A., and Setoyama, H. (1995). Segmented filamentous bacteria are indigenous intestinal bacteria that activate intraepithelial lymphocytes and induce MHC class II molecules and fucosyl asialo GM1 glycolipids on the small intestinal epithelial cells in the ex-germ-free mouse. Microbiol Immunol 39, 555-562.
Umesaki, Y., Setoyama, H., Matsumoto, S., Imaoka, A., and Itoh, K. (1999). Differential roles of segmented filamentous bacteria and clostridia in development of the intestinal immune system. Infect Immun 67, 3504-3511.
Veldhoen, M., Hocking, R. J., Atkins, C. J., Locksley, R. M., and Stockinger, B. (2006). TGFbeta in the context of an inflammatory cytokine milieu supports de novo differentiation of IL-17-producing T cells. Immunity 24, 179-189.
Yen, D., Cheung, J., Scheerens, H., Poulet, F., McClanahan, T., McKenzie, B., Kleinschek, M. A., Owyang, A., Mattson, J., Blumenschein, W., et al. (2006). IL-23 is essential for T cell-mediated colitis and promotes inflammation via IL-17 and IL-6. J Clin Invest 116, 1310-1316.
Zheng, Y., Valdez, P. A., Danilenko, D. M., Hu, Y., Sa, S. M., Gong, Q., Abbas, A. R., Modrusan, Z., Ghilardi, N., de Sauvage, F. J., et al. (2008). Interleukin-22 mediates early host defense against attaching and effacing bacterial pathogens. Nat Med 14, 282-289.
Zhou, L., Lopes, J. E., Chong, M. M., Ivanov, II, Min, R., Victora, G. D., Shen, Y., Du, J., Rubtsov, Y. P., Rudensky, A. Y., et al. (2008). TGF-beta-induced Foxp3 inhibits T(H)17 cell differentiation by antagonizing RORgammat function. Nature 453, 236-240.

What is claimed is:

1-11. (canceled)

12. A method for promoting Th17 differentiation in a subject in need thereof, the method comprising:
a) administering a therapeutic amount of at least one segmented filamentous bacteria (SFB) induced host cell molecule to the subject to enhance Th17 differentiation in the subject.

13. The method of claim 12, further comprising measuring Th17 cell activity in the subject, wherein an increase in the Th17 cell activity in the subject after the administering relative to prior to the administering is a positive indicator of enhanced Th17 differentiation in the subject.

14. The method of claim 13, wherein the increase in the Th17 cell activity is detected as an increase in Th17 cell numbers, Th17 function, or expression of Th17 specific cytokines.

15. The method of claim 14, wherein the increase in Th17 function or expression of Th17 specific cytokines is detected as an increase in expression of at least one of RORγt, IL-17A, IL-17F, IL-22, IL23, IL23R, CD161, and CCR6.

16. The method of claim 13, wherein the increase in Th17 cell activity is measured in a blood sample or biopsy isolated from the subject after the administering and the increase is determined relative to the Th17 activity in a blood sample or biopsy isolated from the subject prior to the administering.

17. The method of claim 13, wherein the subject in need thereof is a patient infected with a pathogenic bacteria or at risk for infection with a pathogenic bacteria.

18. The method of claim 17, wherein the pathogenic bacteria is an antibiotic resistant pathogenic bacteria.

19-29. (canceled)

30. A method for treating a subject infected with a pathogenic bacteria, the method comprising administering to the subject a therapeutically effective amount of at least one SFB induced host cell molecule to the subject, wherein the administering alleviates or prevents symptoms of the pathogenic bacteria-related disorder, thereby treating the pathogenic bacteria-related disorder in the subject.

31. The method of claim 30, wherein the at least one SFB induced host cell molecule is serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; or lymphocyte antigen 6 complex, locus D.

32. The method of claim 30, wherein the at least one SFB induced host cell molecule is administered in a composition comprising the at least one SFB induced host cell molecule and a pharmaceutically acceptable buffer.

33. The method of claim 31, wherein the at least one SFB induced host cell molecule is a combination of at least two SFB induced host cell molecules.

34. The method of claim 12, wherein the at least one SFB induced host cell molecule is serum amyloid A 1; resistin like beta; solute carrier family 6 (neurotransmitter transporter), member 14; placenta expressed transcript 1; serum amyloid A 2; granzyme B.; granzyme A; Z-DNA binding protein 1; nitric oxide synthase 2, inducible, macrophage; hematopoietic cell transcript 1; CD38 antigen; interferon gamma induced GTPase; fucosyltransferase 2; UDP-GlcNAc:betaGal beta-1,3-N-acetylglucosaminyltransferase 7; T-cell receptor gamma, variable 3; sphingomyelin phosphodiesterase, acid-like 3B; betaine-homocysteine methyltransferase; solute carrier family 9 (sodium/hydrogen exchanger), member 3; dual oxidase maturation factor 2; or lymphocyte antigen 6 complex, locus D.

35. The method of claim 34, wherein the at least one SFB induced host cell molecule is a combination of at least two SFB induced host cell molecules.

36. The method of claim 12, wherein the at least one SFB induced host cell molecule is administered in a composition comprising the at least one SFB induced host cell molecule and a pharmaceutically acceptable buffer.
